SEQUENCE LISTING (15 sequences)

SEQ ID NO: 1 - protein, 9 aa, Homo sapiens
  DISULFID (1)..(6)
CYIQNCPLG

SEQ ID NO: 2 - protein, 9 aa, Homo sapiens
  DISULFID (1)..(6)
CYIQNCPLG

SEQ ID NO: 3 - DNA, 512 bp, Homo sapiens
  CDS (37)..(414)
accagtcacggaccctggacccagcgcacccgcaccatggccggccccagcctcgcttgctgtctgctcggcctcctggcgctgacctccgcctgctacatccagaactgccccctgggaggcaagagggccgcgccggacctcgacgtgcgcaagtgcctcccctgcggccccgggggcaaaggccgctgcttcgggcccaatatctgctgcgcggaagagctgggctgcttcgtgggcaccgccgaagcgctgcgctgccaggaggagaactacctgccgtcgccctgccagtccggccagaaggcgtgcgggagcgggggccgctgcgcggtcttgggcctctgctgcagcccggacggctgccacgccgaccctgcctgcgacgcggaagccaccttctcccagcgctgaaacttgatggctccgaacaccctcgaagcgcgccactcgcttcccccatagccaccccagaaatggtgaaaataaaataaagcaggtttttctcctct

SEQ ID NO: 4 - protein, 125 aa, Homo sapiens
MAGPSLACCLLGLLALTSACYIQNCPLGGKRAAPDLDVRKCLPCGPGGKGRCFGPNICCAEELGCFVGTAEALRCQEENYLPSPCQSGQKACGSGGRCAVLGLCCSPDGCHADPACDAEATFSQR

SEQ ID NO: 5 - DNA, 27 bp, Homo sapiens
  CDS (1)..(27)
tgctacatccagaactgccccctggga

SEQ ID NO: 6 - protein, 9 aa, Homo sapiens
CYIQNCPLG

SEQ ID NO: 7 - DNA, 4357 bp, Homo sapiens
  CDS (622)..(1791)
tgttaaggctctgggaccaacgctgggcgaaccagctccgctccggaggggtctgcgcggctggcctcgcccgccccctagcggacccgtgcgatagtgcagcctcagccccaggcacagcgccgcatccagacgccgtccgcgcgcgcagcctgggaggcgctcctcgctcgcctcctgtacccatccagcgaccagccaggctgcggcgaggggattccaaccgaggctccagtgagagacctcagcttagcatcacattaggtgcagccggcaggccatcccaactcgggccgggagcgcacgcgtcactggggccgtcagtcgccgtgcaacttccccggggggagtcaactttaggttcgcctgcggactcggtgcagtggaagccgctgaacatcccgaggaactggcacgctgggggctctgggcttgtggccggtagaggattcccgctcatttgcagtggctcagaggagggtggacccagcagatccgtccgtggagtctccaggagtggagccccgggcgcccctacaccctccgacacgccggatccggcccagccgcgccaagccgtaaagggctcgaaggccggggcgcaccgctgccgccagggtcatggagggcgcgctcgcagccaactggagcgccgaggcagccaacgccagcgccgcgccgccgggggccgagggcaaccgcaccgccggacccccgcggcgcaacgaggccctggcgcgcgtggaggtggcggtgctgtgtctcatcctgctcctggcgctgagcgggaacgcgtgtgtgctgctggcgctgcgcaccacacgccagaagcactcgcgcctcttcttcttcatgaagcacctaagcatcgccgacctggtggtggcagtgtttcaggtgctgccgcagttgctgtgggacatcaccttccgcttctacgggcccgacctgctgtgccgcctggtcaagtacttgcaggtggtgggcatgttcgcctccacctacctgctgctgctcatgtccctggaccgctgcctggccatctgccagccgctgcgctcgctgcgccgccgcaccgaccgcctggcagtgctcgccacgtggctcggctgcctggtggccagcgcgccgcaggtgcacatcttctctctgcgcgaggtggctgacggcgtcttcgactgctgggccgtcttcatccagccctggggacccaaggcctacatcacatggatcacgctagctgtctacatcgtgccggtcatcgtgctcgctacctgctacggccttatcagcttcaagatctggcagaacttgcggctcaagaccgctgcagcggcggcggccgaggcgccagagggcgcggcggctggcgatggggggcgcgtggccctggcgcgtgtcagcagcgtcaagctcatctccaaggccaagatccgcacggtcaagatgactttcatcatcgtgctggccttcatcgtgtgctggacgcctttcttcttcgtgcagatgtggagcgtctgggatgccaacgcgcccaaggaagcctcggccttcatcatcgtcatgctcctggccagcctcaacagctgctgcaacccctggatctacatgctgttcacgggccacctcttccacgaactcgtgcagcgcttcctgtgctgctccgccagctacctgaagggcagacgcctgggagagacgagtgccagcaaaaagagcaactcgtcctcctttgtcctgagccatcgcagctccagccagaggagctgctcccagccatccacggcgtgacccaccagccagggccagggctgcagcctgaggctcaggctgtgctggcataagtgctctgctcctaggtgatggcgtatgtttgtgtataaggtacctatcagtttgtatccctcccctccttggggtggcttcagtggggtggagagtggcctccatgatggaagatgataggggactcagccatcagacaacaccctggcctcctacacgtacttctaccaccctgaacccactgctgccctgggcagtgagtggcttgttttttctcctggacttgtaatttcactccagtatatttttacttcttcattctgggatattgtgaaaagcggtaaatataggattggtgaccaattgggtcaggaagtccagtgttctggacttggggtaagcagtggggttgggacctcagatgggaagggtggtgctaagatcctcctgacctcaaagtgtatttgcctttaagcgaacaaatgctggggtccttggggaccagcttgtcagagggtagccctaagagaaggggattaccttgtaagaccatctggcgcagtggacctattagaacttgggttaaaaatgtttaagaagctaatgtttaagaagcatttgggaaagaaaaagaaataaatgtatccagataggaaaagaagaagtaaaactatttgcagatgacacagttttgtatatagaaaatcctaaggaactcacacacacacacacacacacacacgcacacagctattagaactaataagcaagttccgcaaggtttcaagatacaagatcaatatacaaaaatgaattgtatttctttatactagcaacaaacaatatgaaaacgaagttaaataattccatttataataccatcagaaagaataaaataggaatcaacttaacaaaacaagtgcaagactgaaaactacaaaattggaaagaaattaaagaaggcttaaataaatggaaagacatcctgtgttcatggatcagacttagtattgttaagatggcaatactatcctaactgacatgcagattcagtgcaatccttatgaaaatcatagctggcttttttacagaaattgataagctagtcccaaaattcataaagaaatgcaagggacccagatatccaaataagccttgaaaaagaacaaagttggtggattcacacttcctgatttcataatttacgataaaggtaatcagctcagtgtgttactggtttaaggatagacatacggagcagaataaagagtacagatatgaacacttatacttacggtcaattgatttttgacaaggttcccaagacaattcaatagagaaaggagagtcttttcaacaaatggcaccgagacaatgatatgcaagtgcaaaagaatgaggttggacctttactcacactatgtgcaaaaatcaactcaaaacgcatccaagatctaaatataagagctgaaactataaaatcttagaaagaaacataggcatagatctttgtaaccttgaattaggcagtggtttcttagatatgataccaaagacacaagcaaccaatggaaaaataggtaaattggacttaatcaagatttgaagcttttgtgattgaaaagaccctatcaagaaggtgaaaagataacctgcagaatgggagaaaatatttgcgagtcatatatatgataaggggcttgtatctggaatatataaataactcttataacacaacaataaggagaaaaataaatcaatttaaaaaatgggctaacggtttgaatagacatttctccaaagaagatatgcaaatggctactaagcacatgaaaaatactcaacattattattcattagggaaatgcaagtcaaaatcacaatgagattccagtttacaatcactaggatggctacaataaaaagatggacaagaacgagtgtcggtgaggatgtagagaaactggtagaaatttaaattgttggtgggaatgtaaatggtgcacctgctttgaaaaacagtttggcagtacctcaaaaagttaaacgtagagtgaccatatgacccaggaatgccactcctaggtatttacccaagagaaatgaaaacgtacatacacacaaaaacttgtacaccaatgttcatagcaacattatttgtaatagccaaaaagtggaaacaacccaaatgtctaccaactgatgaatgggaaataaaatgtggtctgtccacgcaatggaacattattagactctaaaaagaaatgaagtactcacacatgccacaacatggatgagccttgaaaacttgctaagtgaaagaagccaggtgcaaaagcccacatattgtctgactgcattgaaatgcaatgtctaaaatggacgaatctatatagagtgaatatagattagcgtttgccagggcctggaggctgtgagagatgaggcatgactactaagggtttggggtttctttttcgggtgatgaaaatgttcgaaattagtggtgattgtgcacgattttgagaatgtactaaaaaccaatgaactttaaaaaataaaaataaacaaa

SEQ ID NO: 8 - protein, 389 aa, Homo sapiens [sequence omitted]

SEQ ID NO: 9 - DNA, 22 bp, Artificial Sequence
  synthetic oligonucleotide
aagatgaccttcatcattgttc

SEQ ID NO: 10 - DNA, 23 bp, Artificial Sequence
  synthetic o oligonucleotide
cgactcaggacgaaggtggagga

SEQ ID NO: 11 - DNA, 24 bp, Artificial Sequence
  synthetic oligonucleotide
gtcaatcctacccccgaagcagct

SEQ ID NO: 12 - DNA, 20 bp, Artificial Sequence
  synthetic oligonucleotide
cagcatgggctccttctcca

SEQ ID NO: 13 - DNA, 23 bp, Artificial Sequence
  synthetic oligonucleotide
cagtgatggcatccactgtggtc

SEQ ID NO: 14 - DNA, 27 bp, Artificial Sequence
  synthetic oligonucleotide
aaggtcggtgtcaacccatttggccgt

SEQ ID NO: 15 - protein, 10 aa, Homo sapiens
  DISULFID (1)..(6)
CYIQNCPLGG

(19) United States
(12) Patent Application Publication
(10) Pub. No.: US 2006/0205636 A1
(43) Pub. Date: Sep. 14, 2006

(54) Oxytocin as cardiomyogenesis inducer and uses thereof

(76) Inventors: Jolanta Gutkowska, Ville Mont-Royal (CA); Joanne Paquin, Montreal (CA); Bogdan A Danalache, Montreal (CA); Marek Jankowski, Montreal (CA)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/518,966

(22) PCT Filed: Jun. 13, 2003
(86) PCT No.: PCT/CA03/00897

§ 371 Date: Nov. 4, 2005

(30) Foreign Application Priority Data
Jun. 21, 2002 (CA) 2,391,118

(51) Int. Cl. A61K 38/12; A61K 48/00

(52) U.S. Cl. 514/9; 514/44

(57) ABSTRACT

The invention relates to oxytocin and oxytocin-related compounds and functional derivatives thereof, and uses thereof to induce differentiation of a non-cardiomyocyte (e.g. a stem/progenitor cell) to a cardiomyocyte. The invention further relates to the methods of prevention or treatment of conditions characterized by or associated with a cardiomyocyte loss or deficiency, by administration of oxytocin or an oxytocin-related compound or a functional derivative thereof to a subject, or via the administration/transplantation of a cell differentiated ex vivo by a method of the invention. The invention further relates to methods, uses, commercial packages and culture media relating to such differentiation and prevention/treatment.

### FIELD OF THE INVENTION

The present invention relates to the field of cell differentiation. More particularly, the present invention relates to the use of oxytocin (OT) as a cell differentiating agent, and even more specifically as an inducer of cardiomyogenesis. The present invention further relates to the use of cardiomyocytes obtained by oxytocin-induced differentiation of non-cardiomyocytes, in the treatment of diseases associated with loss of cardiomyocytes, such as congenital and aging-related heart pathologies.

### BACKGROUND OF THE INVENTION

Each year, up to 7% of the three million newborns in the USA have birth defects, many of which predominantly affect the heart. Furthermore, the great prevalence of cardiovascular diseases in aging populations is well known. In Canada and the US, these diseases account for about 45% of all deaths. A number of drugs exist for the treatment of such diseases. Some drugs are used to improve the cardiodynamic properties of the heart (e.g. agonists/antagonists of adrenergic receptors), while others are used to reduce prejudicing conditions to disease (e.g. substances that attenuate hypercholesterolemia). In some cases, the cardiovascular diseases are treated by surgical intervention.

Today, new prospective therapies envisage myocardial regeneration as an alternative for treating cardiovascular diseases because heart infarction, congestive heart failure and acute myocardial ischemia lead to an irreversible death of cardiac tissue (cardiomyocytes and vascular structures) which becomes replaced by scar tissue. However, there is currently no established cardiac regenerative therapy.

Therefore, there is a need for new prospective therapies and new drugs to prevent and treat heart-related diseases.

### SUMMARY OF THE INVENTION

The present invention pertains to the use of oxytocin (OT), functional derivatives, and/or physiological precursors thereof, and nucleic acids capable of encoding OT, its derivatives and/or prescursors noted above, as a cell differentiating agent and in compositions useful for treating or preventing diseases, such as heart diseases and in particular those associated with loss of cardiomyocytes. More particularly, the present invention pertains to the use of oxytocin or an ocytocin-related compound, corresponding gene construct(s) and/or their functional derivatives as an inducer of cardiomyogenesis, and more specifically as an inducer that promotes the differentiation of non-cardiomyocytes (e.g. stem/progenitor cells) in situ, which can be used to repair, restore or fortify damaged or otherwise inadequate cardiac tissue. The present invention also pertains to the use of oxytocin and functional derivatives thereof to induce cardiac differentiation of non-cardiomyocytes (e.g. stem/progenitor cell) in cell culture in order to provide material for cell or tissue grafting in the heart.

According to a first aspect, the invention provides a method of inducing differentiation of a non-cardiomyocyte into a cardiomyocyte, said method comprising stimulating oxytocin. receptor (OTR) activity in said non-cardiomyocyte. In an embodiment, the method comprises contacting said non-cardiomyocyte with an agent capable of stimulating OTR activity.

In an embodiment, the above-mentioned method is performed in vitro. In an embodiment, the above-mentioned method is performed in vivo.

The invention further provides a method of treating a disease characterized by cardiomyocyte loss or deficiency in an animal, said method comprising stimulating oxytocin receptor (OTR) activity in a non-cardiomyocyte cell of said animal. In an embodiment, the method comprises administering an agent capable of stimulating OTR activity to said animal. In an embodiment, the method comprises admistering a nucleic acid capable of encoding oxytocin or a functional derivative thereof to said animal.

The invention further provides a method of treating a disease characterized by cardiomyocyte loss or deficiency in an animal, said method comprising: inducing, using the above-mentioned method, differentiation of a non-cardiomyocyte cell into a cardiomyocyte; and implanting said cardiomyocyte into said animal. In an embodiment, the method comprises contacting the non-cardiomyocyte with an agent capable of stimulating OTR activity. In an embodiment, the method comprises introducing into the non-cardiomyocyte a nucleic acid capable of encoding oxytocin or a functional derivative thereof. In an embodiment, the non-cardiomyocyte is autologous to said animal. In an embodiment, the method further comprises obtaining said non-cardiomyocyte from said animal prior to inducing said differentiation. In an embodiment, the non-cardiomyocyte is non-autologous to said animal, in a further embodiment, allogenic to said animal, in yet a further embodiment, xenogenic to said animal.

The invention further provides a use of an agent capable of stimulating OTR activity for treating a disease characterized by cardiomyocyte loss or deficiency in an animal.

The invention further provides a commercial package comprising an agent capable of stimulating OTR activity together with instructions for treating a disease characterized by cardiomyocyte loss or deficiency in an animal.

The invention further provides a commercial package comprising an agent capable of stimulating OTR activity together with instructions for inducing differentiation of a non-cardiomyocyte to a cardiomyocyte.

The invention further provides a commercial package comprising a culture medium comprising oxytocin or a functional derivative thereof; together with instructions for culturing a non-cardiomyocyte in said culture medium thereby to induce differentiation of said non-cardiomyocyte into a cardiomyocyte. In an embodiment, the non-cardiomyocyte is a mammalian non-cardiomyocyte, in a further embodiment, a human non-cardiomyocyte. In an embodiment, the oxytocin or functional derivative thereof is present in said medium at a concentration from about 10⁻¹⁰ M to about 10⁻⁴ M, in a further embodiment, from about 10⁻⁹ M to about 10⁻⁶ M, in yet a further embodiment, from about 10⁻⁸ M to about 10⁻⁷ M.

The invention further provides a composition for treatment of a disease characterized by cardiomyocyte loss or deficiency comprising oxytocin or a functional derivative thereof in and a pharmaceutically acceptable carrier.

The invention further provides a method of identifying or characterizing a compound for inducing differentiation of a non-cardiomyocyte into a cardiomyocyte, said method comprising: (a) contacting a test compound with a cell comprising an oxytocin receptor (OTR) or an OTR-encoding nucleic acid; and (b) determining whether OTR activity or expression is increased in the presence of the test compound, said increase in OTR activity or expression being an indication that the test compound may be used for inducing differentiation of a non-cardiomyocyte into a cardiomyocyte.

The invention further provides a method of identifying or characterizing a compound for treatment of a disease characterized by cardiomyocyte loss or deficiency, said method comprising: (a) contacting a test compound with a cell comprising an oxytocin receptor (OTR) or an OTR-encoding nucleic acid; and (b) determining whether OTR activity or expression is increased in the presence of the test compound, said increase in OTR activity or expression being an indication that the test compound may be used for for treatment of a disease characterized by cardiomyocyte loss or deficiency.

The invention further provides a method of identifying a cell capable of differentiation to a cardiomyocyte, said method comprising determining whether OTR activity or expression is present in said cell, said presence being an indication that said cell is capable of differentiation to a cardiomyocyte.

The invention further provides a commercial package comprising means for determining OTR activity or expression together with instructions for identifying a cell capable of differentiation to a cardiomyocyte.

In an embodiment, the above-mentioned agent is selected from the group consisting of oxytocin or a functional derivative thereof, retinoic acid and triiodothyronine (T₃).

In an embodiment, the above-mentioned oxytocin or functional derivative thereof has the structure: 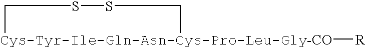
wherein R is selected from the group consisting of OH, NH₂, Gly, Gly-Lys and Gly-Lys-Arg.

In an embodiment, the above-mentioned method comprises introducing into the non-cardiomyocyte a nucleic acid capable of encoding oxytocin or an oxytocin-related compound. In an embodiment, the nucleic acid is selected from the group consisting of: (a) SEQ ID NO:5; (b) a nucleic acid sequence capable of encoding SEQ ID NO:6; and (c) a nucleic acid sequence substantially identical to (a) or (b).

In an embodiment, the non-cardiomyocyte is a mammalian non-cardiomyocyte, in a further embodiment, a human non-cardiomyocyte.

In an embodiment, the non-cardiomyocyte is a stem or progenitor cell. In an embodiment, the stem or progenitor cell is selected from the group consisting of embryonic and adult stem or progenitor cells. In an embodiment, the stem or progenitor cell is selected from the group consisting of circulating and non-circulating stem or progenitor cells.

In an embodiment, the above-mentioned cardiomyocyte is characterized by an alteration of a phenotypic feature relative to said non-cardiomyocyte, wherein said phenotypic feature is selected from the group consisting of: (a) level of oxytocin receptor (OTR) protein or OTR-encoding nucleic acid; (b) level of ANP protein or ANP-encoding nucleic acid; (c) level of muscular MHC protein or MHC-encoding nucleic acid; (d) level of DHPR-alphal protein or DHPR-alphal-encoding nucleic acid; (e) level of sarcomeric marker proteins; (f) level of ion channels; (g) mitochondrial dye retention; (h) appearance of rhythmic beats; and (i) chronotropic responses.

In an embodiment, the above-mentioned animal is a mammal, in a further embodiment, a human.

In an embodiment, the above-mentioned disease is selected from the group consisting of cardiac congenital dysfunctions, aging-related heart pathologies,-heart infarction, congestive heart failure and acute myocardial ischemia.

The invention further provides a pharmaceutical composition which comprises oxytocin and/or of a functional derivative of oxytocin in an amount effective to promote and/or induce differentiation of a non-cardiomyocyte (e.g. stem/progenitor cell) into a cardiomyocyte, and a suitable pharmaceutical acceptable diluent or carrier.

According to another aspect of the invention, oxytocin and/or its functional derivatives, are used as an active agent in the preparation of a medication for preventing or treating a heart disease or for treating an injury to cardiac tissues. The invention also provides methods for preventing or treating a heart disease or for treating an injury to cardiac tissues, comprising the administration to a patient in need thereof of a therapeutically effective amount of oxytocin or of a functional derivative of oxytocin or the administration of a therapeutically effective amount of a composition as defined hereinabove.

According to a further aspect, the invention provides a method for inducing and/or promoting differentiation of cells and more particularly stem/progenitor cells cultured in vitro into cardiac cells, such as cardiomyocytes. In a preferred embodiment, the method comprises the step of providing to the in vitro cultured stem/progenitor cells an effective amount of oxytocin or of a functional derivative thereof. According to another aspect, the present invention provides a method to stimulate the fusion of newly-differentiated cardiomyocytes.

An advantage of the present invention is that it provides effective means for maintaining or stimulating the regeneration of cardiac cells, such as cardiomyocytes, and thereby, it permits the treatment of injuries to the heart tissues. Another advantage of the present invention is that it improves the efficiency of methods for culturing cardiac cells in vitro either as model system or graft material.

Other objects and advantages of the present invention will be apparent upon reading the following non-restrictive description of several preferred embodiments made with reference to the accompanying drawings.

### BRIEF DESCRIPTION OF THE DRAWINGS

FIG. 1 is a diagram showing the time schedule of the differentiation of P19 cells to cardiomyocytes. P19 cells were cultivated as aggregates from day 0 to day 4 in the presence of DMSO (0.5% w/v) or oxytocin (OT) (10⁻⁷ M) as the agent inducing cellular differentiation. At day 4, aggregates (embryoid bodies) were transferred to tissue culture dishes or multiwell plates and grown in the absence of the agent. Micrographs (100× magnification) show undifferentiated cells and day 14 cardiomyocyte derivatives obtained after DMSO or OT treatment.

FIGS. 2A and 2B show that oxytocin (OT) induces myocyte immunological markers in P19 cells. P19 cell aggregates were treated from day 0 to day 4 with DMSO, OT or no differentiation agent, and stained on day 14 with anti-MHC or anti-DHPR-alphal antibodies. FIG. 2A are micrographs (100× magnification) showing day 14 cells that were exposed to OT treatment. Normal light and fluorescence pictures are presented side by side. FIG. 2B is a graph showing immunoreactivity (ir) signals obtained for undifferentiated cells grown in monolayers (Undiff.), non-treated cell aggregates (No inducer) and cell aggregates treated with DMSO or OT. Immunoreactive foci were absent (0), very rare (slightly above zero), or abundant (++0 and +++). Results are representative of 3 independent experiments. Although not presented, aggregates were also treated for 6 days with OT. There was no difference with the 4-day treatment.

FIGS. 3A, 3B and 3C show comparison of the cardiomyogenic effect of oxytocin (OT) and DMSO. FIG. 3A shows the retention of rhodamine¹²³ in non-induced and induced P19 cultures. P19 cells were cultured as aggregates for 4 days in the absence (No inducer) or the presence of OT or DMSO, using 1 petri dish per treatment. At day 4, aggregates of each petri dish were evenly distributed in wells of a 24-well tissue culture plate. At day 8, the cells were incubated for 45 min in the presence of 1 μg/ml of the dye, washed extensively, and cultured in complete medium without dye for 48 h. The photograph shows rhodamine¹²³ retention by cells induced by OT at day 10 of culture. The retained dye was fluorimetrically quantified for each well, and the results are reported as the means±SEM of 24 determinations. The symbol * indicates a highly significant difference with No inducer, and symbol #, a highly significant difference between OT and DMSO treatments (p<0.001). FIG. 3B is a graph showing the time course of appearance of beating cell colonies upon treatment with different agents. Aggregates of 1 petri dish treated for 4 days with the indicated agent(s) were evenly distributed in wells of a 24-well tissue culture plate. Then, each plate was examined at 2-day intervals for the number of wells containing beating cell colonies. The results are representative of 3 independent differentiation experiments. FIG. 3C shows the RT-PCR analysis of ANP gene transcript in undifferentiated and induced cultures. Cell aggregates were exposed to OT or DMSO in the absence or presence of OTA from day 0 to day 4, and RNA was extracted at day 14 of the differentiation protocol. ANP transcript was also evaluated in undifferentiated cells grown in monolayers (Undiff.). Mouse heart ventricle mRNA was used as a positive control. Levels of ANP mRNA were adjusted by dividing by corresponding GAPDH mRNA and then expressed as the percentage of the Undiff. value. Results are reported as the means±SEM of 5 independent studies. The symbol * indicates a significant difference with Undiff., and symbol §, a significant difference between OT and OT+OTA treatments (p<0.05).

FIGS. 4A, 4B, and 4C show that OT and DMSO increase OTR expression in P19 cells. P19 cells were cultured as aggregates for 4 days in the absence (No inducer) or presence of DMSO (0.5%), OT (10⁻⁷ M) and/or OTA (10⁻⁷ M), and then plated in tissue culture dishes where they grew in the absence of the agent. At day 14 of differentiation, the cells were examined for OTR expression, together with undifferentiated (Undiff.) cells grown in monolayers. The results are representative of 3 independent differentiation experiments. FIG. 4A are micrographs showing the immunocytochemistry results. FIG. 4B shows the immunoblotting results (20 μg protein/lane). FIG. 4C shows the RT-PCR analysis.

FIGS. 5A and 5B show results of studies of the concentration dependence of the cardiomyogenic effect of OT. P19 were induced with different concentrations of OT or with DMSO for 4 days and cultured in absence of inducer for 10 more days. (A) Wells were scored for the presence of beating cell colonies. (B) OTR mRNA expression was analyzed on day 14. Results were compared to non-induced (NI) cells.

FIG. 6 shows that P19 cells induced with OT and DMSO produce OT. P19 were induced with OT or DMSO for 4 days and cultured in absence of inducer for 10 more days. Cells were incubated for 4 h in serum-free medium and collected with their culture media for analysis of OT production by RIA.

FIG. 7 shows that OT/OTR system is involved in RA-induced cardiac differentiation of P19 cells. P19 cells were induced with OT (10⁻⁷ M), RA (10⁻⁷ M) or their combination for 4 days, in the absence or presence of OTA (10⁻⁷ M). Culture was pursued for 10 more days in absence of inducer. Wells were scored for the presence of beating cell colonies throughout (A), and OTR mRNA expression analyzed on day 14 (B).

FIG. 8 shows results of studies of morphology of cells at day 6 of differentiation. P19 cells were induced with 10⁻⁸ M RA (a cardiomyogenic concentration) in the absence or presence of 10⁻⁷ M OTA. Morphology was examined two days after the removal of inducer. Cell populations treated with RA+OTA contained some neurons as indicated by the appearance of neuritic processes.

FIG. 9 shows results demonstrating that OT antagonist (OTA) completely inhibits P19 cell differentiation into cardiomyocytes as induced by T₃ as well as by DMSO, retinoic acid (RA). P19 cells were treated for 4 days with 30 nM T₃, 0.5% DMSO or 10⁻⁷ M RA, in the absence or presence of 10⁻⁷ M OTA. Wells were then scored for the presence of beating cell colonies.

FIG. 10 shows results of studies of the in vivo effect of retinoic acid (RA) in the fetal heart (E21). (A) OT concentration measured by radioimmunoassay. (B) OT mRNA as shown by semiquantitative RT-PCR. (C) ¹²⁵I-OTA binding to the fetal sections demonstrated by autoradiography. The representative competition curve of ¹²⁵I-OTA binding to the fetal heart sections by unlabelled OTA. (D) Retinal dehydrogenase 1 transcript detected by Northern blotting in the fetal heart (1) and fetal kidney (2).

FIG. 11 sets forth the DNA sequence (SEQ ID NO:5) of the human oxytocin gene and the encoded polypeptide (SEQ ID NO:6). The signal peptide is underlined. The oxytocin sequence is shown in bold. The remaining C-terminal portion corresponds to neurophysin I. The nucleotide sequence and peptide sequences corresponding to the mature OT peptide are further shown.

FIG. 12 sets forth the DNA sequence (SEQ ID NO:7) of the human oxytocin receptor gene and the encoded polypeptide (SEQ ID NO:8).

### DETAILED DESCRIPTION OF THE INVENTION

The present invention generally pertains to the use of oxytocin or an ocytocin-related compound, corresponding gene construct(s) and/or their functional derivative(s) as a cell-differentiating agent, which may in embodiments be used in compositions useful for treating or preventing heart diseases, in particular those associated with loss of cardiomyocytes. More particularly, the present invention pertains to the use of oxytocin, its gene construct and/or functional derivatives thereof as an inducer of cardiomyogenesis, and more specifically as an inducer that promotes heart regeneration via the differentiation of non-cardiomyocytes into cardiomyocytes (e.g. stem/progenitor cells) in situ. The present invention also pertains to the use of oxytocin or an oxytocin-related compound, corresponding gene construct(s) and/or their functional derivative(s) to induce cardiac differentiation of a non-cardiomyocyte (e.g. a stem/progenitor cell) in cell culture in order to provide material for cell or tissue grafting in the heart. As used herein, the term “non-cardiomyocyte” refers to any cell that lacks at least one phenotypic feature typical of a cardiomyocyte, and is capable of differentiation to a cardiomyocyte. As used herein, the term “stem/progenitor cell” refers to any cell having the capacity of being differentiated into a cell with altered or additional functional characteristics, such as a cardiomyocyte. Stem/progenitor cells include pluripotent stem cells capable of differentiation into a variety of cell types. Preferred stem/progenitor cells contemplated by the present invention are embryonic stem cells, stem cells of developed tissues, and cells of a developed phenotype but still capable of transdifferentiation, i.e. to differentiate to another cell phenotype.

It is demonstrated herein that various agents (e.g. oxytocin, retinoic acid and triiodothyronine) may be used to differentiate a non-cardiomyocyte into a cardiomyocyte. The differentiating activity of these agents is shown to act via stimulation of the oxytocin receptor (OTR), as such differentiation is inhibited in the presense of the OTR antagonist OTA. Thus, it is demonstrated herein that stimulation of OTR activity in a non cardiomyocyte may be used to differentiate the non-cardiomyocyte to a cardiomyocyte. Accordingly, the invention provides a method of differentiating or inducing differentiation of a non-cardiomyocyte to a cardiomyocyte, the method comprising stimulating or inducing OTR activity in the non-cardiomyocyte. In an embodiment, the method comprises contacting the non-cardiomycoyte with an agent capable of stimulating or inducing OTR activity. “An agent capable of stimulating/inducing OTR activity” as used herein refers to any agent which when introduced to or contacted with an appropriate system or cell, results in a stimulation of OTR activity in that system or cell. In embodiments, such agents include OTR agonists (e.g. oxytocin or related compounds or functional derivatives thereof) as well as other varieties of agents capable of such stiumulation, such as retinoic acid and triiodothyronine.

The present invention thus provides a novel cell differentiating agent and more particularly a new cardiomyogenic factor. As used herein, “cardiomyogenic factor” refers to any compound (or to any mixture of compounds) that promotes the genesis, maturation, growth, and regeneration of cardiac cells, and more specifically promotes differentiation of a non-cardiomyocyte (e.g. a stem/progenitor cell) into a cardiomyocyte.

A cardiomyocyte may be distinguished from a non-cardiomyocyte based on an alteration of a variety of phenotypic features, including, but not limited to the level of oxytocin receptor (OTR) protein or OTR-encoding nucleic acid; level of ANP protein or ANP-encoding nucleic acid; level of muscular/cardiac myosin heavy chain (MHC) protein or MHC-encoding nucleic acid; level of dihydropyridine receptor-alphal (DHPRαl) protein or DHPRαl-encoding nucleic acid; level of sarcomeric marker proteins; level of ion channels; mitochondrial dye retention; appearance of rhythmic beats and chronotropic responses.

In an embodiment, the present invention describes the use of oxytocin in a pharmaceutical composition and in a method for promoting the genesis, maturation, growth, and regeneration of cardiac cells. The cardiac cells that are most susceptible to benefit from the composition of the invention are stem cells and newly differentiated cardiomyocytes. Also, the present invention relates to the use of oxytocin for the preparation of a composition or a medicament for the treatment or prevention of heart diseases and in particular those associated with loss of cardiomyocytes.

The pharmaceutical composition of the invention thus comprises oxytocin and/or of a functional derivative oxytocin in an amount effective to promote and/or induce differentiation of stem/progenitor cells into cardiac cells, and a suitable pharmaceutical acceptable diluent or carrier.

Oxytocin is a nonapeptide with two cysteine residues that form a disulfide bridge between positions 1 and 6 and corresponds to the formula: 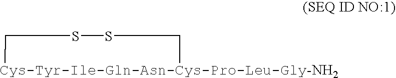
The non-(carboxy-terminal) amidated version has the following structure: 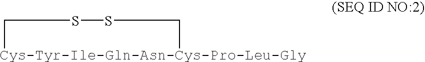
Another version of OT retains an additional C-terminal Gly residue (retained from the proprotein), and has the following structure.

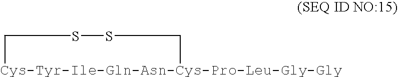

Thus, the oxytocin or an ocytocin-related compound, and/or their functional derivative(s) according to the present invention are in embodiments substantially pure oxytocin produced by chemical synthesis, or purified from plasma and various tissues, but preferably from the pituitary gland, or produced by recombinant techniques. As generally understood and used herein, the term substantially pure refers to an oxytocin preparation that is generally lacking cellular or other undesirable components.

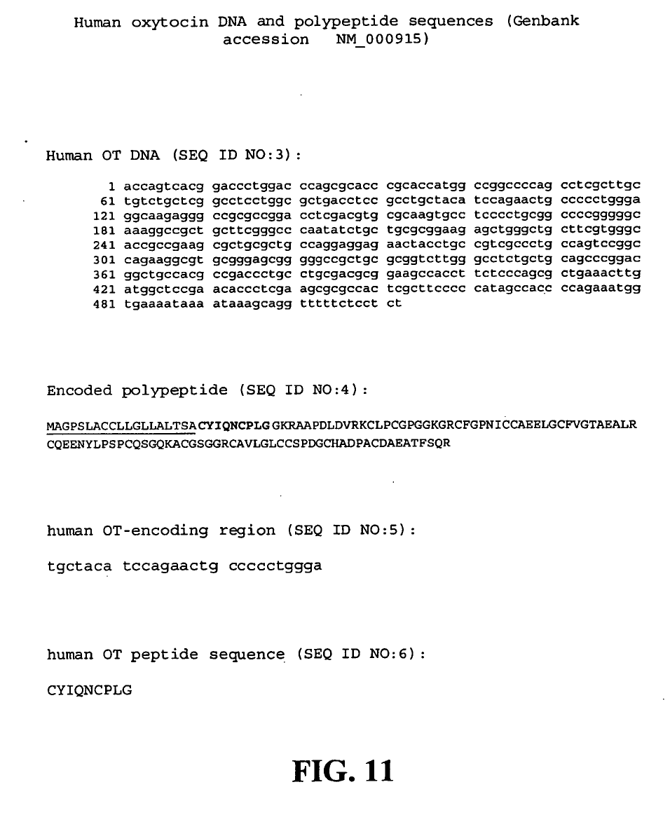

“oxytocin-related compound” or “oxytocin-like compound” refers to a compound which is structurally and/or functionally related to oxytocin. Such compounds include homologs, variants or fragments of oxytocin which retain oxytocin activity. Such compounds may comprise a peptide which is substantially identical to oxytocin or fragment thereof, e.g. substantially identical to the human oxytocin peptide set forth above and in FIG. 11. Similarly, such compounds include peptides or proteins encoded by a nucleic acid sequence which is substantially identical to, or is related by hybridization criteria (see below) to a nucleic acid sequence capable of encoding oxytocin, such as the region of human oxytocin DNA set forth in FIG. 11. Such compounds further include precursors (e.g. naturally-occurring precursors) or prodrugs which are metabolized or otherwise converted to an active compound at the site of action.

A “functional derivative”, as is generally understood and used herein, refers to a polypeptide sequence that possesses a functional biological activity that is substantially similar to the biological activity of the whole protein sequence. A functional derivative of a protein may or may not contain post-translational modifications such as covalently linked carbohydrate, or disulphide bonds, if such modification is not necessary for the performance of a specific function. The term “functional derivative” is intended to the “fragments”, “segments”, “variants”, “analogs” or “chemical derivatives” of a protein.

Oxytocin-related compounds, also include precursors which can be modified to produce mature, active oxytocin, or an analog having oxytocin activity. In this regard, the human oxytocin gene encodes two proteins, oxytocin and neurophysin I. Oxytocin is therefore naturally produced as a proprotein of oxytocin-neurophysin I, as shown in FIG. 11 (human oxytocin gene DNA [SEQ ID NO:3] and encoded polypeptide [SEQ ID NO:4]; Genbank accession NM_{—}00915.2), which is subsequently modified to the active oxytocin peptide. With reference to the DNA sequence in FIG. 11, the regions of the sequence correspond to the following:

| | DNA region |
|---|---|
| coding sequence nucleotides | 37-414 |
| signal peptide | 37-93 |
| proprotein | 94-411 |
| mature oxytocin peptide | 94-120 |
| mature neurophysin I peptide | 130-408 |

With reference to the polypeptide sequence of FIG. 11, the signal peptide is underlined and the sequence of oxytocin is shown in bold.

The terms “fragment” and “segment” as are generally understood and used herein, refer to a section of a protein, and are meant to refer to any portion of the amino acid sequence.

The term “variant” as is generally understood and used herein, refers to a protein that is substantially similar in structure and biological activity to either the protein or fragment thereof. Thus two proteins are considered variants if they possess a common activity and may substitute each other, even if the amino acid sequence, the secondary, tertiary, or quaternary structure of one of the proteins is not identical to that found in the other.

The term “analog” as is generally understood and used herein, refers to a protein that is substantially similar in function to oxytocin. Preferred OT analogs include for instance extended forms of OT such as OT-Gly, OT-Gly-Lys and OT-Gly-Lys-Arg. These extended forms are biological oxytocin precursors in vivo.

As used herein, a protein is said to be a “chemical derivative” of another protein when it contains additional chemical moieties not normally part of the protein, said moieties being added by using-techniques well known in the art. Such moieties may improve the protein solubility, absorption, bioavailability, biological half life, and the like. Any undesirable toxicity and side effects of the protein may be attenuated and even eliminated by using such moieties. For example, OT and OT fragments can be covalently coupled to biocompatible polymers (polyvinyl-alcohol, polyethylene-glycol, etc) in order to improve stability or to decrease antigenicity.

The amount of oxytocin and/or functional derivatives thereof present in the composition of the present invention is a therapeutically effective amount. A therapeutically effective amount of oxytocin is that amount of oxytocin or derivative thereof necessary so that the protein acts as a cardiomyogenic factor, and more particularly the amount necessary so that the protein promote the generation, maturation, growth, and restoration of cardiac cells, and more specifically, cardiomyocytes, and the fortification of cardiac tissue with such cells. The exact amount of oxytocin and/or functional derivatives thereof to be used will vary according to factors such as the protein biological activity, the type of condition being treated as well as the other ingredients in the composition. Typically, the amount of oxytocin should vary from about 10⁻¹⁵ M to about 10⁻² M. In a preferred embodiment, oxytocin is present in the composition in an amount from about 10⁻¹⁰ M to about 10⁴ M, preferably from about 10⁻⁹ M to about 10⁻⁶ M. In the embodiments, the composition comprises about 10⁻⁷ of oxytocin for in vivo applications and 10⁻⁶ M of oxytocin for ex vivo applications.

A “therapeutically effective amount” refers to an amount effective, at dosages and for periods of time necessary, to achieve the desired therapeutic result, such as reduction of progression of a disease characterized by cardiomyocyte loss or deficiency. A therapeutically effective amount of oxytocin, or an oxytocin-related compound, may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing or inhibiting onset or progression of a disease characterized by cardiomyocyte loss or deficiency. A prophylactically effective amount can be determined as described above for the therapeutically effective amount. For any particular subject, specific dosage regimens may be adjusted over time according to the individual need and the professional judgement of the person administering or supervising the administration of the compositions.

Further therapeutic agents can be added to the composition of the invention. For instance, the composition of the invention may also comprise therapeutic agents such as modulators of heart function (agonists/antagonists of adrenergic receptors, activators of neurohormones, cytokines, signaling second messengers such as cAMP/cGMP/calcium or their analogs, inhibitors of the degradation of second messengers); growth factors, steroid/glucocorticoid/retinoid/thyroid hormones which modulate heart gene expression; proteases/protease inhibitors/cell adhesion proteins/angiogenic factors that modulate cardiac tissue organization and/or vascularization; antioxidants that provide cell protection to endogenous cardiac tissue as well as to exogenous cardiomyocyte cultures before, during and after engrafting; anticoagulants; immunosuppressive drugs.

Further to the therapeutic agents, the pharmaceutical compositions of the invention may also contain metal chelators (proteinic or not), metal scavengers (proteinic or not), coating agents, preserving agents, solubilizing agents, stabilizing agents, wetting agents, emulsifiers, sweeteners, colorants, odorants, salts, buffers, coating agents and/or antioxidants. For preparing such pharmaceutical compositions, methods well known in the art may be used.

The method of preparation of the composition of the invention consists simply in the mixing of purified oxytocin and other component(s) in a suitable solution in order to get a homogenous physiological suspension. A suitable solution is an isotonic buffered saline solution comprising sodium, potassium, magnesium or manganese, and calcium ions at physiological concentrations, that is it mimics the ion composition of the extracellular fluid. The solution has an osmotic pressure varying from 280 to 340 mOsmol, and a pH varying from 7.0 to 7.4. The buffered saline solution can be selected from the group consisting of Krebs-Henseleit's, Krebs-Ringer's or Hank's buffer, as examples.

The composition of the invention could be suitable to treat and/or prevent diseases such as cardiovascular diseases or treat an injury to heart tissues. Cardiovascular diseases which could be treated include cardiac congenital malformations (e.g. cardiac atrophy, cardiac hypertrophy, defective cardiac chamber organization) or dysfunctions that could be caused by stress conditions during the fetal life or at birth, including ischemic conditions, infections by microorganisms, exposure to teratogenic toxicants, substances or drugs. Cardiovascular diseases which could be treated also include aging-related heart pathologies, such as heart infarction, congestive heart failure, and acute myocardial ischemia.

The composition could also be involved in modulating heart development during embryogenesis by inducing cardiomyogenesis. The composition of the invention may thus be administered during gestation to correct development of the heart.

The composition of the invention may be administered alone or as part of a more complex pharmaceutical composition according the desired use and route of administration. For instance, the composition of the invention could comprise a vector, such as a plasmid or a virus, comprising a DNA sequence coding for native oxytocin, coding for a modified/fusion oxytocin protein having an increased cardiomyogenic activity, or an increased stability. Anyhow, for preparing such compositions, methods well known in the art may be used.

Oxytocin and/or its derivatives may be coupled to a biocompatible polymer (e.g. polyethylene glycol, polyvinyl alcohol) to reduce antigenicity when administered parenterally.

The composition of the invention and/or more complex pharmaceutical compositions comprising the same may be given via various routes of administration. For instance, the composition may be administered in the form of sterile injectable preparations, for example, as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intramuscularly or sub-cutaneously by injection or by infusion. The composition may also be administered per os (e.g. capsules), nasal spray, transdermal delivery (e.g. iontophoresis). Suitable dosages will vary, depending upon factors such as the amount of each of the components in the composition, the desired effect (fast or long term), the disease or disorder to be treated, the route of administration and the age and weight of the individual to be treated.

In an embodiment, the composition of the invention and/or more complex pharmaceutical compositions comprising the same may be given by direct injection into the heart at the site of infarction or injury. Indeed, damaged sites were shown to attract newly added cardiomyocytes or progenitor cells.

Oxytocin or a functional derivative thereof could also be used in methods for culturing cardiac cells in vitro. By providing an effective amount of oxytocin to in vitro cultured stem/progenitor cells, it will induce the differentiation of the cultured stem/progenitor cells into cardiac cells, such as cardiomyocytes, and then will promote the aggregation of cardiac cells and promote the tissular organization of in vitro cultured heart tissues. Oxytocin or a functional derivative thereof could thus be very useful for providing cardiac tissues for transplant purposes.

Therefore, a related aspect of the invention relates to a method for inducing cardiomyogenic differentiation from cells, such as non-cardiomyocytes (e.g. stem cells), i.e. to induce differentiation of a non-cardiomyocyte to a cardiomyocyte. In a preferred embodiment, the method comprises the step of contacting the non-cardiomyocyte with an effective amount of oxytocin or an oxytocin-related compound or functional derivatives thereof. The method may in embodiments comprise introducing into a cell a nucleic acid (e.g. in a suitable vector) capable of encoding oxytocin or an oxytocin-related compound or functional derivative thereof. According to another aspect, the present invention provides a method to stimulate the fusion of newly-differentiated cardiomyocytes. The cells are contacted with about 10⁻¹⁰ M to about 10⁻⁴ M of OT, preferably from about 10⁻⁹ M to about 10⁻⁶ M of OT, in embodiments for about 8 h to about 14 days.

The invention further provides a culture medium to induce differentiation of non-cardiomyocytes cultured therein to cardiomyocytes. The culture medium comprises oxytocin or an oxytocin-related compound, or a functional derivative thereof. The oxytocin may in embodiments be present in the medium at a concentration equal to or greater than about 10⁻¹⁰ M, in a further embodiment of about 10⁻¹⁰ M to about 10⁻⁴ M, in a further embodiment from about 10⁻⁹ M to about 10⁻⁶ M, in yet a further embodiment from about 10⁻⁸ M to about 10⁻⁶ M (in embodiments, about 10⁻⁷M of oxytocin for in vivo applications and and about 10⁻⁶ M of oxytocin for ex vivo applications). The medium may further comprise various standard media components and elements conducive to cell culture, such as salts, acids and bases to control ionic strength and pH of the medium, antibiotics to reduce contamination, and any other elements or factors conducive to cell culture. Such standard media components are known in the art and are commercially avialable. The medium may be provided in liquid or powder form, and is in an embodiment substantially sterile. The medium may be provided in a commercial package together with instructions for differentiation of a non-cardiomyocyte to a cardiomyocyte. The invention further provides a commercial package comprising a standard or base medium, oxytocin or an oxytocin-related compound, or functional derivatives thereof, together with instructions for differentiation of a non-cardiomyocyte to a cardiomyocyte. The invention further provides a commercial package comprising oxytocin or an oxytocin-related compound, or a functional derivative thereof, together with instructions for differentiation of a non-cardiomyocyte to a cardiomyocyte.

It will be understood by one skilled in the art that the methods and compositions contemplated by the present invention when applicable, may advantageously be used either in vitro, ex vivo and/or in vivo.

With regard to increasing or upregulating expression of a oxytocin in a cell, various methods of introducing oxytocin-encoding nucleic acids into the cell may be used, examples of which are described below. Methods such as the gene therapy methods discussed below may be used in this regard. Examples of oxytocin-encoding nucleic acids include the nucleic acid of SEQ ID NO:5, a nucleic acid capable of encoding the polypeptide of SEQ ID NOs: 1,2 or 6, or nucleic acids substantially identical thereto. The method may also comprise administering to an area or cardiac tissue a cell comprising such an oxytocin-encoding nucleic acid, via for example implantation or introduction of such a cell comprising such a oxytocin-encoding nucleic acid.

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “homologous” to another sequence if the two sequences are substantially identical and the functional activity of the sequences is conserved (as used herein, the term ‘homologous’ does not infer evolutionary relatedness). Two nucleic acid sequences are considered substantially identical if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%. As used herein, a given percentage of homology between sequences denotes the degree of sequence identity in optimally aligned sequences. An “unrelated” or “non-homologous” sequence shares less than 40% identity, though preferably less than about 25% identity, with a sequence of interest.

Substantially complementary nucleic acids are nucleic acids in which the “complement” of one molecule is substantially identical to the other molecule. Optimal alignment of sequences for comparisons of identity may be conducted using a variety of algorithms, such as the local homology algorithm of Smith and Waterman,1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridization to filter-bound sequences under moderately stringent conditions may, for example, be performed in 0.5 M NaHPO₄; 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH.

The invention further provides a composition for the prevention and/or treatment of a disease characterized by cardiomyocyte loss or deficiency comprising oxytocin or an oxytocin-related compound, in admixture with a pharmaceutically acceptable carrier.

The invention further provides a use of oxytocin or an oxytocin-related compound, or the above-mentioned composition, for the prevention and/or treatment of a disease characterized by cardiomyocyte loss or deficiency.

The invention further provides a use of oxytocin or an oxytocin-related compound for the preparation of a medicament for the prevention and/or treatment of a disease characterized by cardiomyocyte loss or deficiency.

The invention further provides commercial packages comprising an oxytocin, or an oxytocin-related compound, or the above-mentioned composition together with instructions for the prevention and/or treatment of a disease characterized by cardiomyocyte loss or deficiency.

In accordance with another aspect of the invention, therapeutic compositions of the present invention, comprising oxytocin, or an oxytocin-related compound, may be provided in containers or commercial packages which further comprise instructions for their use for the prevention and/or treatment of a disease characterized by cardiomyocyte loss or deficiency.

Given that induction of oxytocin receptor (OTR) activity results in an induction of differentiation of a non-cardiomyocyte to a cardiomyocyte as shown herein, compounds which are capable of modulating OTR expression or activity can be used to induce differentiation of a non-cardiomyocyte to a cardiomyocyte and/or for the prevention and treatment of disease characterized by cardiomyocyte loss or deficiency. Therefore, the invention further relates to screening methods for the identification and characterization of compounds useful for the induction of differentiation of a non-cardiomyocyte to a cardiomyocyte and/or for the prevention and treatment of disease characterized by cardiomyocyte loss or deficiency.

Therefore, the invention further provides a method of identifying or characterizing a compound for inducing differentiation of a non-cardiomyocyte cell into a cardiomyocyte, or for treatment of a disease characterized by cardiomyocyte loss or deficiency, said method comprising:
(a) contacting a test compound with a cell comprising an oxytocin receptor (OTR) or an OTR-encoding nucleic acid; and
(b) determining whether OTR activity or expression is increased in the presence of the test compound, said increase in OTR activity or expression being an indication that the test compound may be used for inducing differentiation of a non-cardiomyocyte cell into a cardiomyocyte, or for treatment of a disease characterized by cardiomyocyte loss or deficiency.

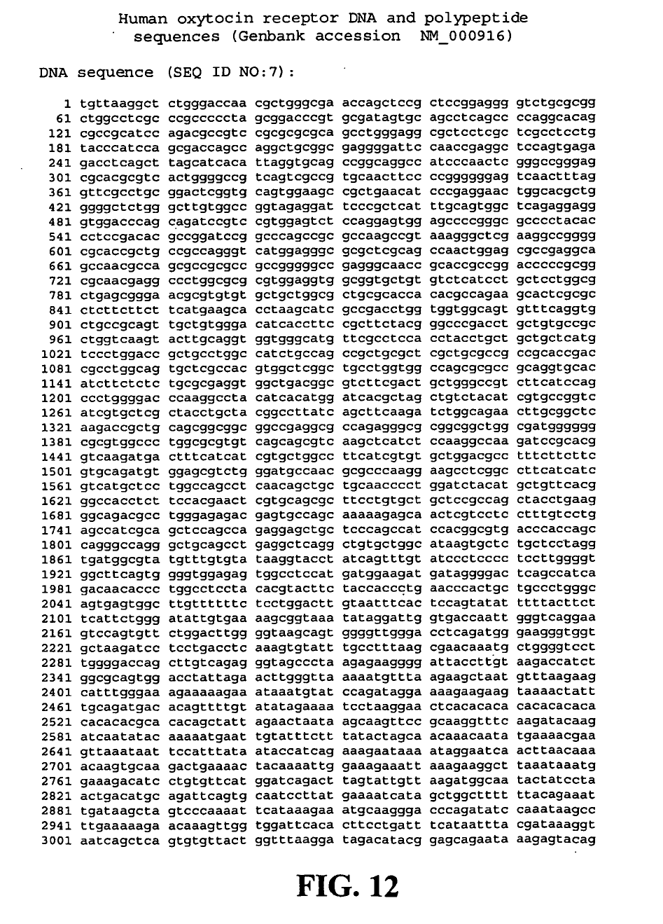

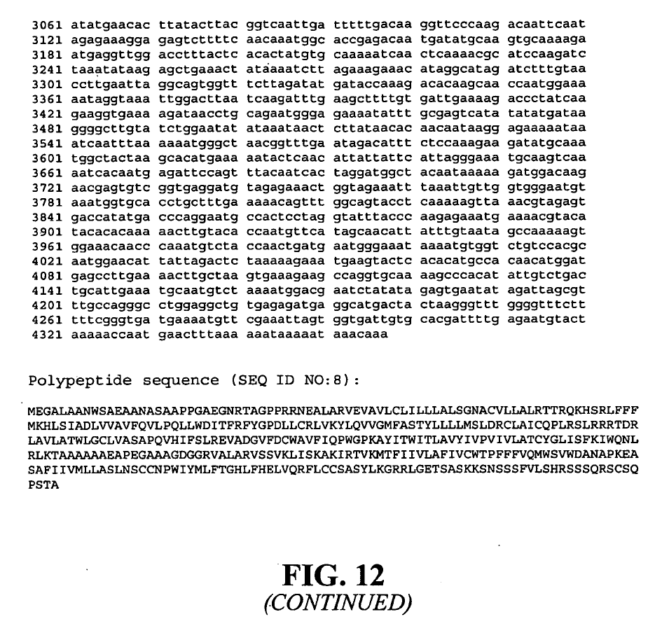

In an embodiment, the above-mentioned cell comprising OTR is a cell which comprises endogenous levels or expression of OTR. The OTR-comprising cell may be an appropriate host cell in which an exogenously source of OTR was introduced. Such a host cell may be prepared by the introduction of nucleic acid sequences encoding OTR into the host cell under providing conditions for the expression of an OTR. In an embodiment, such a nucleic acid is DNA. Such host cells may be prokaryotic or eukaryotic, bacterial, yeast, amphibian or mammalian. In an embodiment, such host cells are human. DNA and polypeptide sequences corresponding to human OTR are for example set forth in FIG. 12 and SEQ ID NOs: 7 and 8.

The invention further provides a method of identifying or characterizing cells or progenitor cells capable of or having the potential to differentiate into cardiomyocytes, the method comprising determining whether such a cell expresses OTR and/or responds to OT by increasing OTR expression, wherein OTR expression and/or such a response to OT indicate that the cell is capable of or has the potential to differentiate into a cardiomyocyte. In embodiments, the latter method may be used in conjuntion with a differentiation method of the invention, whereby a cell is first identified as being capable of differentiation, and is subsequently differentiated into a cardiomyocyte using the methods and agents described herein.

OTR expression may be measured on the transcriptional or translational level, e.g. by the amount of RNA or protein produced. OTR expression can thus be evaluated by RT-PCR, immunocytochemistry, Western blotting (Paquin et al., PNAS 2002) and/or binding studies. RNA may be detected by for example Northern analysis or by the reverse transcriptase-polymerase chain reaction (RT-PCR) method (see for example Sambrook et al (1989) Molecular Cloning: A Laboratory Manual (second edition), Cold Spring Harbor Laboratory Press, Cold Spring Harbor, N.Y., USA). Protein levels may be detected either directly using affinity reagents (e.g. an antibody or fragment thereof [for methods, see for example Harlow, E. and Lane, D (1988) Antibodies: A Laboratory Manual, Cold Spring Harbor Laboratory Press, Cold Spring Harbor, N.Y.]; a ligand which binds the protein), or by measurement of the protein's activity or a related detectable phenotype.

The above-mentioned method may be employed either with a single test compound or a plurality or library (e.g. a combinatorial library) of test compounds. In the latter case, synergistic effects provided by combinations of compounds may also be identified and characterized. The above-mentioned compounds may be used for prevention and/or treatment of a disease characterized by cardiomyocyte loss or deficiency, or may be used as lead compounds for the development and testing of additional compounds having improved specificity, efficacy and/or pharmacological (e.g. pharmacokinetic) properties. In certain embodiments, one or a plurality of the steps of the screening/testing methods of the invention may be automated.

In another aspect of the invention, the isolated nucleic acid, for example a nucleic acid sequence encoding oxytocin or a homolog, fragment or variant thereof, may further be incorporated into a recombinant expression vector. In an embodiment, the vector will comprise transcriptional regulatory element or a promoter operably-linked to an oxytocin-coding region. A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since for example enhancers generally function when separated from the promoters by several kilobases and intronic sequences may be of variable lengths, some polynucleotide elements may be operably-linked but not contiguous. “Transcriptional regulatory element” is a generic term that refers to DNA sequences, such as initiation and termination signals, enhancers, and promoters, splicing signals, polyadenylation signals which induce or control transcription of protein coding sequences with which they are operably-linked. The recombinant expression may further encode additional sequences, such as signal peptide sequences to allow entry of the encoded polypeptide into the secretory pathway, and other domains, which may provide added stability to the polypeptide or are conducive to isolation/purification of the peptide produced (e.g. fusions with commonly used domains/peptides/affinity tags).

The recombinant expression vector of the present invention can be constructed by standard techniques known to one of ordinary skill in the art and found, for example, in Sambrook et al. (1989) in Molecular Cloning: A Laboratory Manual. A variety of strategies are available for ligating fragments of DNA, the choice of which depends on the nature of the termini of the DNA fragments and can be readily determined by persons skilled in the art. The vectors of the present invention may also contain other sequence elements to facilitate vector propagation and selection in bacteria and host cells. In addition, the vectors of the present invention may comprise a sequence of nucleotides for one or more restriction endonuclease sites. Coding sequences such as for selectable markers and reporter genes are well known to persons skilled in the art. An example of suitable vector is the HSV amplicon-based vector described in the Examples below.

A recombinant expression vector comprising a nucleic acid sequence of the present invention may be introduced into a host cell, which may include a living cell capable of expressing the protein coding region from the defined recombinant expression vector. The living cell may include both a cultured cell and a cell within a living organism. Accordingly, the invention also provides host cells containing the recombinant expression vectors of the invention. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (Molecular Cloning: A Laboratory Manual, 2nd Edition, Cold Spring Harbor Laboratory press (1989)), and other laboratory manuals. Methods for introducing DNA into mammalian cells in vivo are also known, and may be used to deliver the vector DNA of the invention to a subject for gene therapy for a disease characterized by cardiomyocyte loss or deficiency.

A cell, tissue, organ, or organism into which has been introduced a foreign nucleic acid, is considered “transformed”, “transfected”, or “transgenic”. A transgenic or transformed cell or organism also includes progeny of the cell or organism and progeny produced from a breeding program employing a transgenic organism as a parent and exhibiting an altered phenotype resulting from the presence of a recombinant nucleic acid construct. A transgenic organism is therefore an organism that has been transformed with a heterologous nucleic acid, or the progeny of such an organism that includes the transgene.

For stable transfection of mammalian cells, it is known that, depending upon the expression vector and transfection technique used, only a small fraction of cells may integrate the foreign DNA into their genome. In order to identify and select these integrants, a gene that encodes a selectable marker (such as resistance to antibiotics) may be introduced into the host cells along with the gene of interest. Preferred selectable markers include those that confer resistance to drugs, such as G418, hygromycin and methotrexate. Nucleic acids encoding a selectable marker may be introduced into a host cell on the same vector as that encoding the peptide compound or may be introduced on a separate vector. Cells stably transfected with the introduced nucleic acid may be identified by drug selection (cells that have incorporated the selectable marker gene will survive, while the other cells die).

Given that administration of oxytocin induces differentiation of a non-cardiomyocyte to a cardiomyocyte, as described herein, a further aspect of the present invention is the treatment of a disease characterized by cardiomyocyte loss or deficiency by administering to a subject a nucleic acid molecule encoding oxytocin or an oxytocin-related compound. Suitable methods of administration include gene therapy methods.

A nucleic acid of the invention may be delivered to cells in vivo using methods such as direct injection of DNA, receptor-mediated DNA uptake, viral-mediated transfection or non-viral transfection and lipid based transfection, all of which may involve the use of gene therapy vectors. Direct injection has been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo may be used. Such an apparatus may be commercially available (e.g., from BioRad). Naked DNA may also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson el al. (1992) J. Biol. Chem. 267:963-967; and U.S. Pat. No. 5,166,320). Binding of the DNA-ligand complex to the receptor may facilitate uptake of the DNA by receptor-mediated endocytosis. A DNA-ligand complex linked to adenovirus capsids which disrupt endosomes, thereby releasing material into the cytoplasm, may be used to avoid degradation of the complex by intracellular lysosomes (see for example Curiel el al. (1991) Proc. Natl. Acad. Sci. USA 88:8850; Cristiano et al. (1993) Proc. Natl. Acad. Sci. USA 90:2122-2126).

Defective retroviruses are well characterized for use as gene therapy vectors (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include .psi.Crip, .psi.Cre, .psi.2 and .psi.Am. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

For use as a gene therapy vector, the genome of an adenovirus may be manipulated so that it encodes and expresses a peptide compound of the invention, but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin el al. (1992) Proc. Natl. Acad. Sci. USA 89:2581-2584).

Adeno-associated virus (AAV) may be used as a gene therapy vector for delivery of DNA for gene therapy purposes. AAV is a naturally occurring defective virus that requires another virus, such as an adenovirus or a herpes virus, as a helper virus for efficient replication and a productive life cycle (Muzyczka et al. Curr. Topics in Micro. and Immunol. (1992) 158:97-129). AAV may be used to integrate DNA into non-dividing cells (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). An AAV vector such as that described in Tratschin et al. (1985) Mol. Cell. Biol. 5:3251-3260 may be used to introduce DNA into cells (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790). Lentiviral gene therapy vectors may also be adapted for use in the invention.

General methods for gene therapy are known in the art. See for example, U.S. Pat. No. 5,399,346 by Anderson et al. A biocompatible capsule for delivering genetic material is described in PCT Publication WO 95/05452 by Baetge et al. Methods of gene transfer into hematopoietic cells have also previously been reported (see Clapp, D. W., et al., Blood 78: 1132-1139 (1991); Anderson, Science 288:627-9 (2000); and Cavazzana-Calvo et al., Science 288:669-72 (2000)).

The invention further relates to implantation, transplantation and grafting methods, to introduce into a subject a cell comprising a nucleic acid capable of encoding oxytocin or an oxytocin-related compound. The nucleic acid may be present in a vector as described above, the vector being introduced into the cell in vitro, using for example the methods described above. In an embodiment, the cell is autologous, and is obtained from the subject. In embodiments, the cell is non-autologous, e.g. allogenic or xenogenic.

### EXAMPLES

Oxytocin (OT), a nonapeptide largely expressed in the hypothalamus, has long been recognized as a female reproductive hormone necessary for uterine contraction during parturition, timing and amplification of labour, milk ejection during lactation, and ovulation (1). However, the last decades have shed new light on OT functions. It has been shown that both sexes have equivalent concentrations of OT in the hypophysis and plasma as well as a similar number of oxytocinergic neurons in the hypothalamus (2), and respond to the same stimuli for OT release (3, 4). It also appears that reproductive functions and maternal behaviour are preserved in OT^{-/-} mutant mice (5). Both OT^{-/-} males and females are fertile, and females are capable of parturition although they lack the milk ejection reflex (5, 6). These observations indicate that OT is not essential for reproduction, and data now underline the involvement of OT in sexual behaviour, cognition, memory, tolerance, adaptation, food and water intake, and cardiovascular functions (1, 7, 8).

Recently, a new role has been suggested for OT as a growth and cellular differentiation factor. The antiproliferative effect of OT, mediated by OT receptors (OTR), has been documented in breast cancer cells (9) and other tumors (10-12). In contrast to its effect on tumoral cells, a mitogenic action of OT has also been described. OT stimulates the proliferation of thymocytes (13, 14) and mitotic activity in the prostate epithelium (15), vascular endothelium (16) and trophoblasts (17) . OT has also been reported to enhance myoepithelial cell differentiation and proliferation in the mouse mammary gland (18). The possibility that OT has trophic effects on the embryo has not been investigated intensively. However, OT has been shown to have an influence on the developing heart: OT administered in excess to the fetus may impair cardiac growth in humans and rats (19, 20), and OTR suppression by specific OT antagonists (OTA) in the early stage of chicken egg development leads to cardiac malformation in the embryos (21). It is not known whether the trophic effects of OT on the heart are direct or indirect.

OT's indirect actions could be related to its cardiovascular functions observed in adult rats (7, 22-24). Indeed, we uncovered the entire OT/OTR system in the rat heart, and showed that cardiac OTR activation is coupled to the release of atrial natriuretic peptide (ANP), a potent diuretic, natriuretic and vasorelaxant hormone that is also involved in cell growth regulation (7, 8). A role for ANP in cardiomyogenesis has even been suggested by Cameron et al. (25). In support of a potential action of OT on cardiac development, a maximal OT protein level was seen in the heart at day 21 of gestation and postnatal days 1-4, when cardiac myocytes are at a stage of intense hyperplasia (26).

The P19 mouse embryonal carcinoma cell line is an established model of cell differentiation. Developmentally, pluripotent P19 cells give rise to the formation of cell derivatives of all 3 germ layers (27) (28) and appear to differentiate via the same mechanisms as normal embryonic stem cells (27, 29). When cultured in the presence of 10⁻⁶ M retinoic acid (RA), a physiologically-relevant morphogen, P19 cells efficiently (≧95%) differentiate to neurons (27, 30, 31). The solvent DMSO induces cardiac differentiation, albeit not as efficiently (≦15%) (27, 32). DMSO has been shown to activate essential cardiogenic transcription factors, such as GATA-4 and Nkx-2.5 (32, 33). However, the mechanisms responsible for triggering these genes in the embryo are still unknown, as is the mode of action of DMSO with respect to the cardiomyogenic program in P19 cells.

In the present example, the inventors investigated whether OT induces differentiation of P19 cells into a cardiomyocyte phenotype. The results confirm that OT has a potential naturally-occurring cardiomorphogen activity.

### Example 1

### Materials and Methods

Culture and Differentiation of P19 Cells

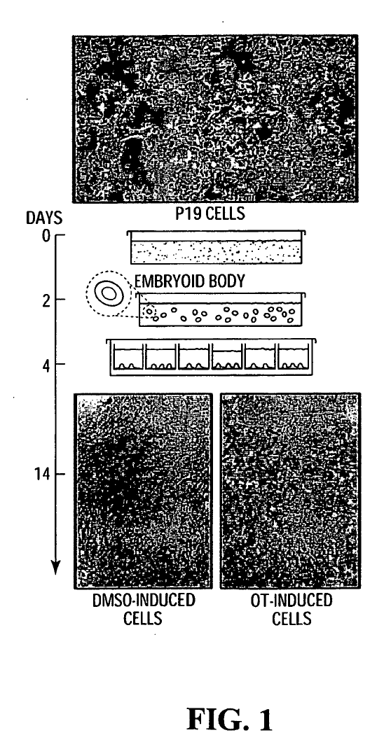

P19 cells were propagated and differentiated according to the procedures of Rudnicki and McBurney (28), with minor modifications. Undifferentiated cells were propagated in complete medium containing α-modified Eagle's minimal essential medium (α-MEM; GIBCO-BRL Burlington, Ontario, Canada) supplemented with 2.5% heat-inactivated fetal bovine serum, 7.5% heat-inactivated donor bovine serum (Cansera International, Rexdale, Ontario, Canada), and the antibiotics (GIBCO-BRL) penicillin G (50 U/ml) and streptomycin (50 μg/ml). The cultures were maintained at 37° C. in a humidified atmosphere of 5% CO₂ and passaged every 2 days. The general protocol used for differentiation of P19 cells is depicted in FIG. 1.

Differentiation was routinely induced with DMSO. Briefly, 0.25×10⁶ cells were allowed to aggregate for 4 days in non-adhesive bacteriological grade petri dishes (6-cm diameter) containing 5 ml complete medium, in the presence of 0.5% (v/v) DMSO (Sigma Chemical Co., St. Louis, Mo.). At day 2 of aggregation, the inducing culture medium was replenished. At day 4, aggregates were transferred to tissue culture grade vessels (10-cm diameter dishes or 24/48-well plates), and cultured in complete medium in the absence of differentiation-inducing agent. Aggregation was also done in the absence of DMSO, and in the presence of 10⁻⁷ M OT and/or 10⁻⁷ OTA ([d(CH₂)₅¹,Tyr(Me)²,Thr⁴,Orn⁸,Tyr-NH₂⁹]-vasotocin), both from Peninsula Laboratories Inc. (San Carlos, Calif.). The cell populations were analyzed at days 10-14 of the entire differentiation protocol, at a time cardiac cells normally beat synchronously.

Cell Morphology, Staining and Immunocytochemistry

Examinations were done under a Zeiss® inverted microscope (Zeiss IM, Carl Zeiss, Jena, Germany) equipped with phase-contrast objectives, filters for rhodamine and fluorescein fluorescence, a MC 100® camera and a photoautomat unit. Micrographs were taken with Kodak Technical Pan® film (for cell morphology) or with Kodak T-Max 400® or Elite-II 100® film (for fluorescence).

For morphological examination, cells were grown directly onto the plastic surface of tissue culture vessels. For staining with rhodamine¹²³ (Sigma), day-4 aggregates were distributed in 24-well culture plates and grown until day 8. Then, dye was added to the culture medium at a final concentration of 1 μg/ml for 45 min, and afterwards, the cells were washed extensively with phosphate-buffered saline (PBS) and cultured for 48 h in the absence of the dye. Dye retained by cells in each well was measured by a fluorescence microplate reader (SPECTRA Max Gemini®, Molecular Devices, Sunnyvale, Calif.) at 505 nm for excitation and 534 nm for emission.

For immunocytofluorescence studies, cells were grown onto glass coverslips coated with 0.1% gelatin. They were then fixed by 20-min incubation in PBS containing 4% paraformaldehyde, rinsed in PBS and stored at 4° C. in this buffer until used. All subsequent steps of permeabilization, washing and incubation with antibodies were performed at room temperature. Fixed cells were permeabilized for 10 min in PBS containing 0.005% saponin, blocked for 60 min in PBS-BSA-saponin (PBS containing 1% bovine serum albumin and 0.005% saponin), incubated for 45 min with the primary antibody diluted 1/50 and for 45 min with a fluorescein-conjugated swine anti-goat IgG antibody (Biosource International, Camarillo, Calif.) diluted 1/1000. PBS-BSA-saponin was used for washing between incubations and antibodies were diluted in the same buffer but containing 1.5% normal swine serum (Jackson Immuno Research Laboratories Inc., West Grove, Pa.). Coverslips were mounted in PBS containing 50% glycerol, and immediately examined under the microscope. The primary antibodies were all from Santa Cruz Biotechnology, Inc. (Santa Cruz, Calif.) and produced in goat: antibody C-20 against OT receptor (OTR), antibody K-16 against sarcomeric myosin heavy chain (MHC), and antibody N-19 against dihydropyridine receptor-alphal (DHPR-alphal).

Analysis of OT by Radioimmunoassay(RIA)

P19 cells induced with DMSO or OT were cultured until day 14 in absence of inducer. Cells were washed carefully with PBS and incubated for 4 h in serum-free α-MEM supplemented with protease inhibiors (aprotinin, 30 μg/mL and soybean trypsin inhibitor (SBTI), 100 μg/mL). Cells and media were collected, extracted on Sep-Pak C₁₈-cartridges (Waters Canada, Mississauga, Ontario), lyophilized, resuspended in a minimal volume of α-MEM and assayed by RIA as described (8). RIA was also done on undiluted serum combination used for P19 cell culture (1 part of fetal bovine serum and 3 parts of donor bovine serum).

Analysis by Reverse Transcription-Polymerase Chain Reaction (RT-PCR)

Total cellular RNA was extracted with TRIzol® Reagent (Invitrogen Life Technologies, Burlington, Ontario, Canada), and poly(A)⁺mRNA was affinity purified from 200 μg of total RNA onto Oligotex® mRNA columns (Qiagen, Mississauga, Ontario, Canada), as per the manufacturers' instructions. First-strand cDNA was synthesized in a final volume of 40 μl containing first-strand buffer, 3 μg of cellular RNA, 4 μl of hexanucleotide primers (Amersham-Pharmacia, Baie d'Urfé, Quebec, Canada), and avian myeloblastosis virus reverse transcriptase (12 units/μg RNA; Invitrogen). First-strand cDNA (5 μl) was then used for PCR amplification with OTR, ANP or GAPDH exon-specific oligonucleotide primers in a Robocycler Gradient 40 thermocycler (Stratagene, La Jolla, Calif.). Sequences of mouse OTR and ANP genes have been described (26, 34). Conditions for RT-PCR analysis of mouse OTR were adapted from Wagner et al. (6, 7). For all PCR studies the number of cycles used was within the linear range of amplification. The OTR sense and antisense primers were respectively the 22-bp 5′-AAGATGACCTTCATCATTGTTC-3′ (SEQ ID NO:9) and the 23-bp 5′-CGACTCAGGACGAAGGTGGAGGA-3′ (SEQ ID NO:10). Amplification was performed over 32 cycles, each involving 1 min at 94° C., 1.5 min at 62° C. and 1.5 min at 72° C., and was terminated by a 5-min final extension at 72° C. The ANP antisense and sense primers were respectively the 24-bp 5′-GTCAATCCTACCCCCGAAGCAGCT-3′ (SEQ ID NO:11) and the 20-bp 5′-CAGCATGGGCTCCTTCTCCA-3′ (SEQ ID NO:12). Amplification was performed over 25-30 cycles, each involving 1 min at 94° C., 1 min at 65° C. and 3 min at 72° C., and was terminated by a 5-min final extension at 72° C. The amplification of GAPDH mRNA, a constitutively and ubiquitously expressed gene, served as an internal standard for RT-PCR analysis. The 23-bp antisense primer 5′-CAGTGATGGCATCCACTGTGGTC-3′ (SEQ ID NO:13) and the 23-bp sense primer 5′-AAGGTCGGTGTCAACCCATTTGGCCGT-3′ (SEQ ID NO:14) were used. Amplification was performed over 23 cycles, each involving 1 min at 94° C., 1.5 min at 59° C. and 2 min at 72° C.

Western Blot Analysis

Cells were collected by scraping, homogenized in sucrose buffer (20 mM Hepes/Tris, pH 7.4, containing 250 mM sucrose and 20 μg/ml of the protease inhibitor phenylmethylsulfonyl fluoride), then centrifuged at 3000 g for 10 min at 4° C. to remove debris. The supernatants were centrifuged at 100 000 g for 45 min at 4° C., and the pellets were resuspended in sucrose buffer for analysis of protein content by a modified Bradford assay (30). Aliquots (20 μg protein) were subjected to polyacrylamide gel electrophoresis in the presence of sodium dodecyl sulfate (SDS-PAGE) under reducing conditions (35) followed by electrotransfer onto pure nitrocellulose membrane (Hybond-C; Amersham-Pharmacia); Molecular size calibration was achieved using Broad Standard Solution (Bio-Rad Laboratories Ltd., Mississauga, Ontario, Canada). The nitrocellulose blots were blocked overnight with 5% nonfat milk in Tris-buffered saline (TBS: 20 mM Tris.Cl, pH 8.0, 140 mM NaCl, 1% BSA and 0.1% Tween-20), then probed with goat C20 antibody (anti-OTR; 1/1,000) for 2 h at room temperature. Antibody incubations and washes were performed in TBS throughout. Detection was realized by enhanced chemiluminescence with an Amersham-Pharmacia ECL® kit and an appropriate peroxidase-conjugated secondary antibody (29). Autoluminograms were developed in an AFP Imaging Mini-med 190® X-Ray Film Processor (AFP Corp., Elmsford, N.Y.).

Statistics

Results are reported as the mean values ± SEM. Comparisons between treatments were done by unpaired Student's t test.

### Example 2

### Effect of OT or DMSO Treatment on P19 Cells

Using the time schedule depicted in FIG. 1, treatment of P19 cell aggregates with 10⁻⁷ M OT induced the formation of rhythmically-beating cells resembling primary cardiomyocytes isolated from the heart of newborn animals. A similar phenotypic change was already reported for treatment with 0.5-1% DMSO (27, 28, 30, 32). Aggregates treated with OT or DMSO were observed to have a 1.5-fold smaller mean diameter than their untreated counterparts (data not shown), a finding that could reflect the antimitotic activity of OT and DMSO.

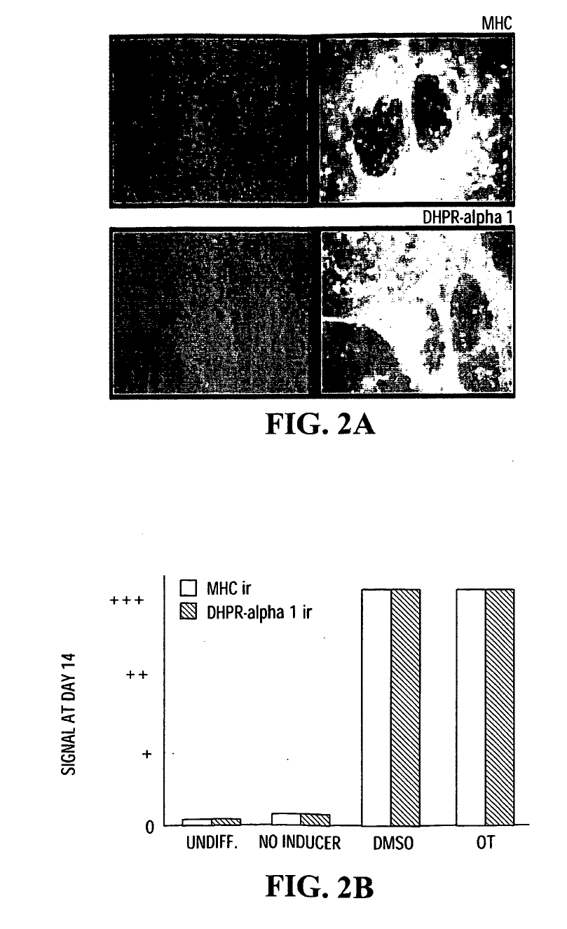

We examined whether treatment of cell aggregates with OT induced the expression of the cardiac muscle markers sarcomeric MHC and DHPR-alphal. Sarcomeric MHC is expressed in contractile muscular cells as is DHPR-alphal, a component of intracellular junctions critical for the coupling of excitation-contraction (27, 32, 36). As presented in FIG. 2B, undifferentiated cells were negative for MHC, as reported (27, 28, 32), and for DHPR-alphal. However as with DMSO, OT induced the appearance of numerous, intense, immunoreactive foci in cell populations (FIGS. 2A, B). In both cases, there were cell subpopulations that did not respond positively (FIG. 2A) and seemed to be mainly undifferentiated cells according to morphological criteria. We and others have shown that undifferentiated cells remain in DMSO-treated P19 cultures by probing for Stage-specific Embryonic Antigen-1, an established marker of the undifferentiated state (27, 28, 30). Cell aggregates not exposed to OT or DMSO were not positive for MHC and DHPR-alphal although they sometimes showed very rare and small immunoreactive foci (FIG. 2B, No inducer). This occasional staining could be due to spontaneous differentiation events triggered by high cell densities such as those encountered in aggregates (27, 28).

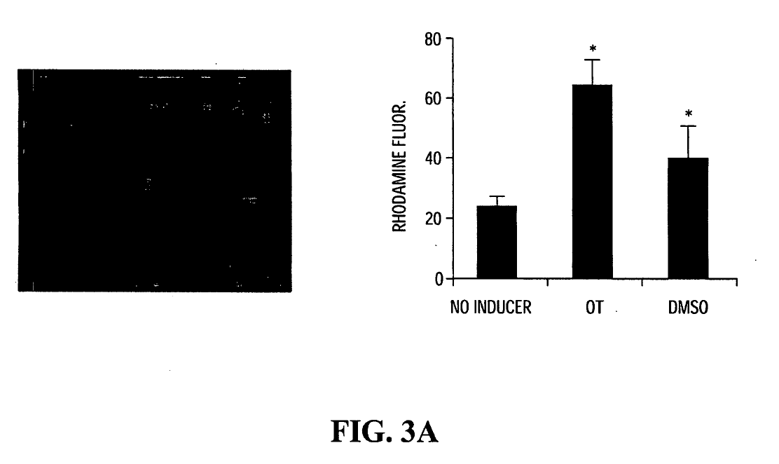

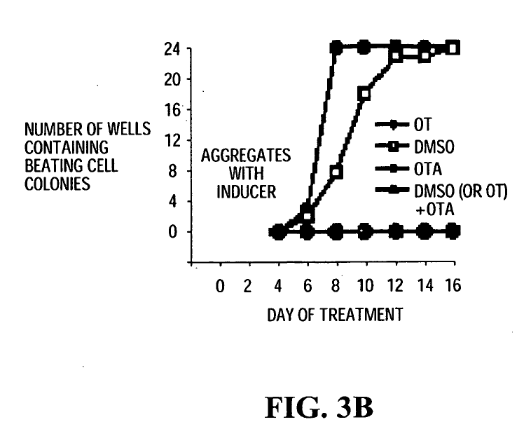

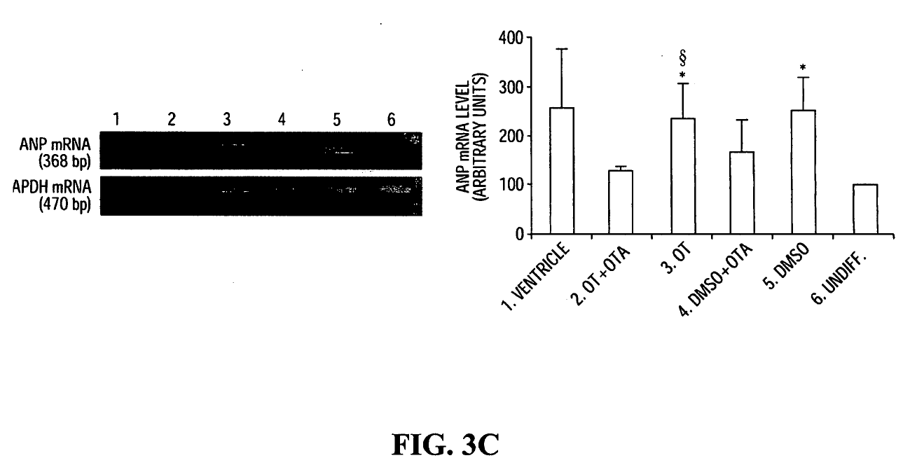

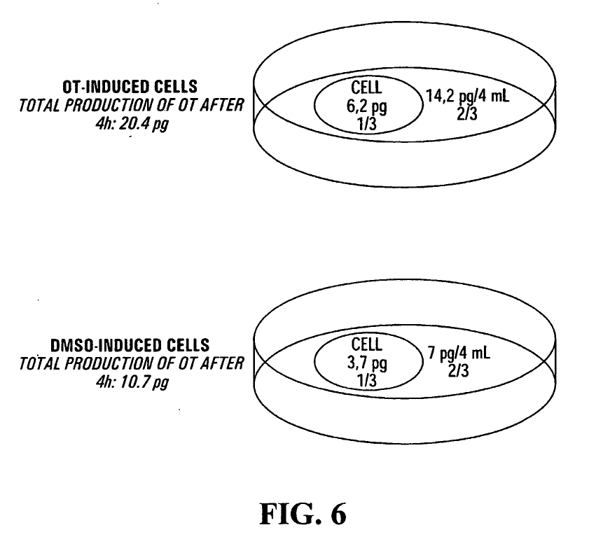

We also compared the cardiogenic potency of OT and DMSO. First, potency was simply quantitated by rhodamine¹²³ retention in cells, taking advantage of the fact that this dye, which penetrates all cell types, is retained for much longer periods (days instead of hours) in cardiac cells than in other cell types (37). To meet their energy requirements for muscular contraction, cardiomyocytes have indeed abundant mitochondria, the cell organelles that accumulate rhodamine¹²³. FIG. 3A shows that exposure of the cell aggregates to OT and DMSO significantly increased cellular retention of the dye by 2-3 fold compared to non-induced aggregates (p<0.001), and this increase at day 10 of differentiation was even significantly higher after OT than DMSO treatment (p<0.001). Since P19-derived cardiomyocytes beat in culture, we also compared the time course of appearance of beating cells after treatment of aggregates with DMSO or OT. We found that OT stimulated the production of beating cell colonies in all 24 independently growing cultures by day 8 whereas the same result was obtained in cells induced by DMSO only by day 12 (FIG. 3B). The cardiogenic action of OT was specific and receptor-mediated, since no beating cells were seen when 10⁻⁷ M OTA was used in place of OT or in combination with OT (FIG. 3B). Interestingly, OTA also abolished the cardiogenic action of DMSO (FIG. 3B). Analysis of OT production by RIA also confirmed cardiomyocyte functionning. Like normal cardiomyocytes, P19 cells induced with DMSO and OT release OT in their culture medium (FIG. 6). Finally, cardiogenic potency was evaluated via ANP expression since this peptide is abundantly produced by cardiomyocytes. The results showed that at day 14 of differentiation ANP mRNA level was significantly upregulated in OT-treated P19 aggregates as compared to undifferentiated cells (p<0.05), and this upregulation was at similar level after DMSO treatment (FIG. 3C). As for cell beating, OTA prevented OT-induced upregulation of ANP expression (FIG. 3C, p<0.05). Although the effect of OTA on DMSO-induced ANP expression was not statistically significant, the inhibitory tendency was observed in all experiments (FIG. 3C). The inhibitory action of OTA on DMSO cardiomyogenic properties was thus more evident by the beating than the ANP criteria. Altogether, rhodamine¹²³ absorption, and the time-course formation of beating cells and abundance of ANP mRNA pointed to a potent cardiomyogenic effect of OT. In addition, the cardiomyogenic action of OT and even that of DMSO appear to involve OTR.

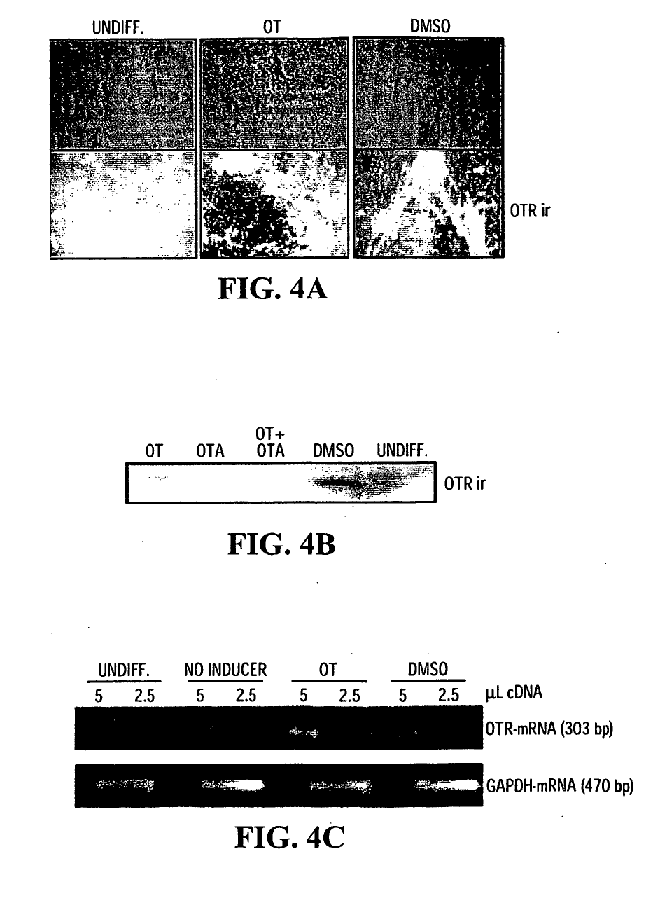

To further investigate the involvement of OTR in cardiomyogenesis, we examined OTR expression in P19 cells. OTR protein (FIGS. 4A, B) and mRNA (FIG. 4C) were present at low levels in undifferentiated cells, indicating that these cells can respond minimally to OT. OTR expression remained at low levels in aggregates not exposed to OT or DMSO (FIG. 4C, No inducer). In contrast, intense OTR immunoreactive foci were observed in cell populations after OT or DMSO treatment (FIG. 4A). These findings corresponded to the results of Western blotting (FIG. 4B) and RT-PCR analysis of OTR (FIG. 4C), both indicating increased OTR expression. In accordance with the absence of a cardiomyogenic effect of OTA and the inhibitory action of OTA on OT-induced cardiac differentiation, OTA did not upregulate OTR expression by itself and inhibited OT-induced OTR upregulation (FIG. 4B). Thus, the OTR-dependent cardiogenic effect of OT and DMSO seems to involve upregulation of OTR expression.

As described herein, it is shown that OT added, to the culture medium of P19 stem cell aggregates induced cardiomyogenic differentiation, which was demonstrated by monitoring the expression of MHC, DHPR-alphal and ANP cardiac markers, production of OT, retention of a mitochondrial-specific dye and the appearance of beating cell colonies. The cardiogenic effect of OT was specific and mediated by OTR because it was abolished by OTA. OT also upregulated OTR expression. These results suggest a new role for the OT/OTR system in heart genesis and development.

The P19 cell line is an excellent cell differentiation model that mimics the events of early cardioembryogenesis. Differentiation of P19 cells to cardiomyodytes by aggregation and exposure to OMSO was shown to be associated with induction of the cardiac-specific subtype of endothelin receptors (38). In addition, brain natriuretic peptide and ANP were observed in newly-formed striated muscle structures upon DMSO treatment and not in undifferentiated P19 cells and their neuronal derivatives (39). In the studies described herein, DMSO- and OT-induced ANP transcript levels reached about 5-10% of that found in the adult mouse atrium—the richest site of ANP synthesis. Several transcription factors having an essential role in cardiogenesis are upregulated in DMSO-induced P19 cells. This was shown to be the case for the zinc-finger containing GATA-4, the homeobox gene Nkx2-5, and the myocyte enhancer factor 2C (32, 33, 40), and the overexpression of either factor in P19 cells was sufficient to induce cardiac differentiation in the absence of DMSO (32, 41, 42). Little is known about the molecular mechanisms underlying the activation of these genes, but DMSO was found to increase intracellular Ca²⁺ levels and was suspected to affect a pathway that has an extracellular component, possibly serum-borne (27, 43, 44). Interestingly, the data described herein indicate that OTR are upregulated to a similar extent by OT and DMSO, and other studies have reported that OTR function modulates intracellular Ca²⁺ concentration in some cell types (1). It is thus tempting to suggest that OT could be a serum-borne factor that is active in DMSO-induced differentiation.

One of the mechanisms by which OT and DMSO trigger cardiac differentiation involves OTR since both agents upregulated the expression of this receptor, and OTA totally abolished their cardiomyogenic action as well as prevented OT-stimulated effect on OTR expression. Homologous regulation of OTR expression by OT itself was observed in the brain and in astroglial cell cultures (46, 47). It is noteworthy that, like DMSO, RA, used at low levels (10⁻⁸-10⁻⁹ M), induces cardiac differentiation of P19 cells (27, 28). This observation could have some relevance to the OT/OTR system since RA was shown to upregulate OT expression in the fetal heart (26).

Several studies have proposed a role for OT as a growth and differentiation/maturation factor in a gestational/perinatal context. In the mother, OT is required for postpartum alveolar proliferation, and induces differentiation and proliferation of myoepithelial cells of the mammary gland necessary for milk ejection (1, 18). The OT/OTR system is expressed in human cumulus/luteal cells surrounding oocytes and weak OTR gene expression is even observed in oocytes (48). Moreover, when fertilized mouse oocytes are cultured with OT in vitro, they develop at a higher rate into the blastocyst stage than their unstimulated counterparts (48). Spontaneous myometrial contractures are known to occur during pregnancy in sheep and controlled contractures induced by application of OT pulses to pregnant ewes have been shown to accelerate fetal cardiovascular function (49).

All these studies thus strongly suggest involvement of the maternal and embryonal OT/OTR systems in development of the embryo, and our work points to a particular involvement of OT in the priming of cardiogenesis. We think that OT could also assist the maturation of newly-differentiated cardiomyocytes by stimulating their fusion since beating cells derived from OT-induced P19 cells formed fiber-like structures. Such a fusogenic action was recently reported for OT on skeletal myoblasts in vitro (50). Our results may find application in therapies that consider the replacement of cardiac tissue lost after injury. In this context, OT could be used as a trophic factor to assist the compensatory division of myocytes shown to occur in infarcted organs (51), or to prime the cardiomyogenesis of a variety of progenitor/stem cells to be grafted in the injured heart (52, 53).

### Example 3

### Studies of the Mechanism of Differentiation Mediated by OT/OTR System

Materials and Methods

Induction of P19 Cells with Different OT Concentrations, RA and Trilodothyronine (T₃)

P19 cells were subjected to the cardiac differentiation protocol depicted in Example 1, except that different concentrations of OT were used during the four days of aggregation. At day 4, aggregates were transferred to 24-well plates and scored for the appearance of beating cell colonies. In addition, cells were collected at day 14 for analysis of OTR or OT expression by RT-PCR. Cell aggregates were also treated with RA and/or T₃.

OT RT-PCR

RT-PCR for OT was Performed with Three Different Pairs of Primers Covering the Entire Coding Sequence of OT Gene (8).

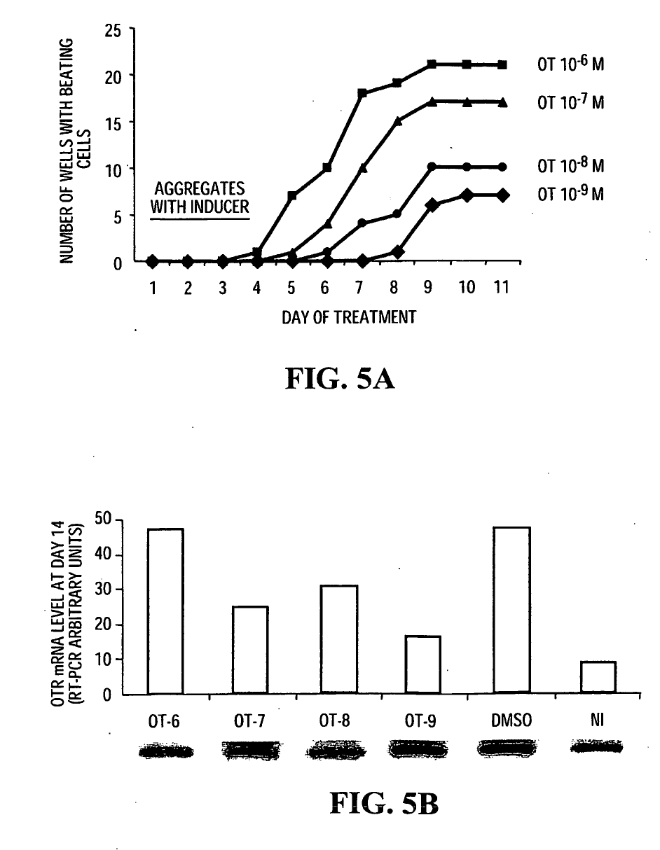

OT differentiation of P19 cells into rhythmically beating cardiomyocytes is concentration-dependent within the range of 10⁻⁶ to 10⁻⁹ M (FIG. 5). OTR mRNA expression in differentiated populations (at day 14) also supports the concentration-dependent effect (FIG. 5). Serum was recently recognized as a potent OTR stimulator in various cells (reviewed by Kimura et al. 2003). We have data showing that OT concentation in fetal and donor bovine sera (used in P19 cell cultures) is 10⁻¹¹ M. Consequently, the final OT concentration of 10⁻¹² M in the culture medium is not sufficient to induce cardiac differentiation of P19 cells. It is possible that some exogenously supplied compounds, such as DMSO, can induce cardiomyocyte differentiation in conditions of reduced OT concentration (below 10⁻⁹ M) by increasing OTR affinity for OT, by upregulating OTR expression and/or by inducing the same signaling transducers as OT. Serum-borne factors could also participate in DMSO action.

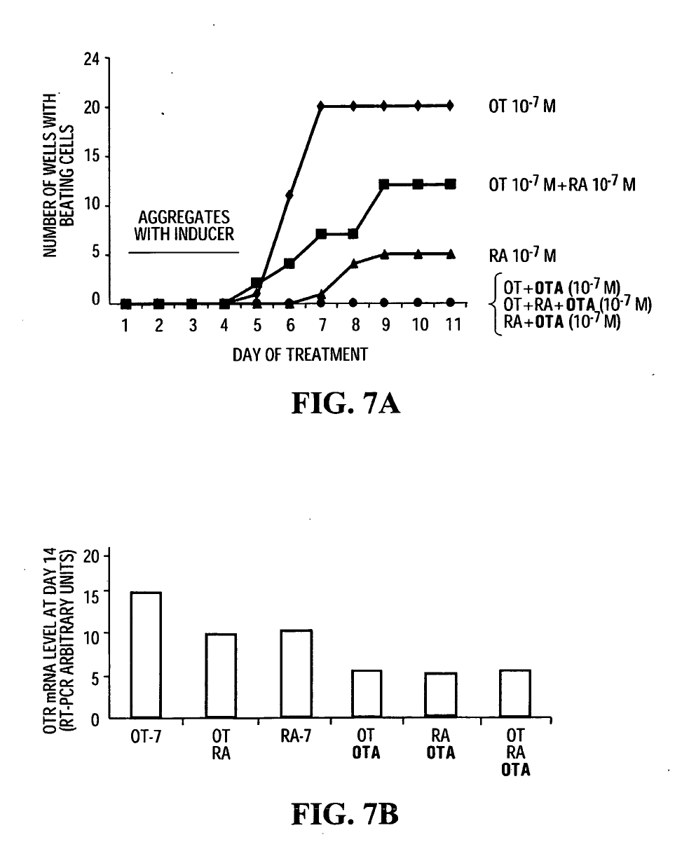

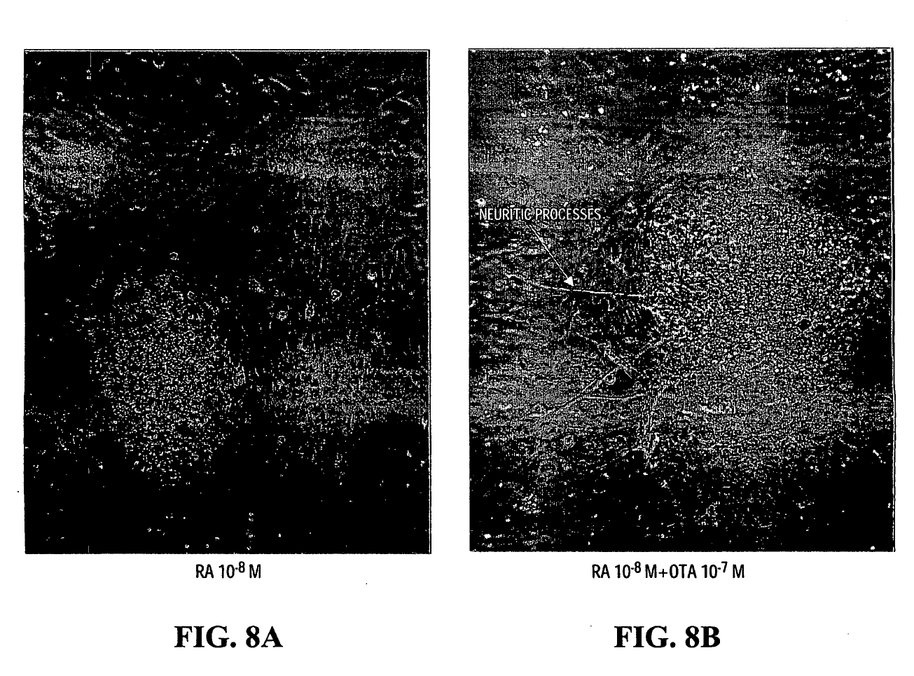

Retinoic acid (RA), a very well known cardiac morphogen, stimulated P19 cell differentiation into cardiomyocytes at concentration of 10⁻⁷ M, as reported previously (27,28). This is demonstrated by the appearance of rhythmically beating cells and OTR upregulation (FIG. 7). As in the case of OT and DMSO (Paquin et al., PNAS 2002), OTA completely inhibits RA-induced cardiac differentiation (FIG. 7). This new result suggests that OT/OTR system is involved in the cardiomyogenic action of RA. We noticed that the presence of OTA in cultures induced with 10⁻⁸ M RA (a cardiomyogenic concentration) results in the apparition of some neural cells (FIG. 8). The neurogenic potential of RA is known in P19 model but is realized at higher concentration (10⁻⁶ M; 27-31). This observation suggests that OT and RA pathways cross-talk during cell differentiation.

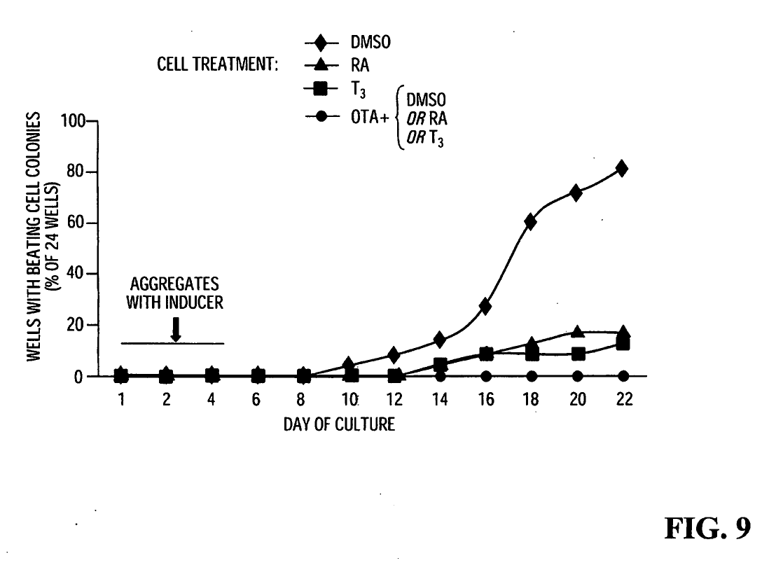

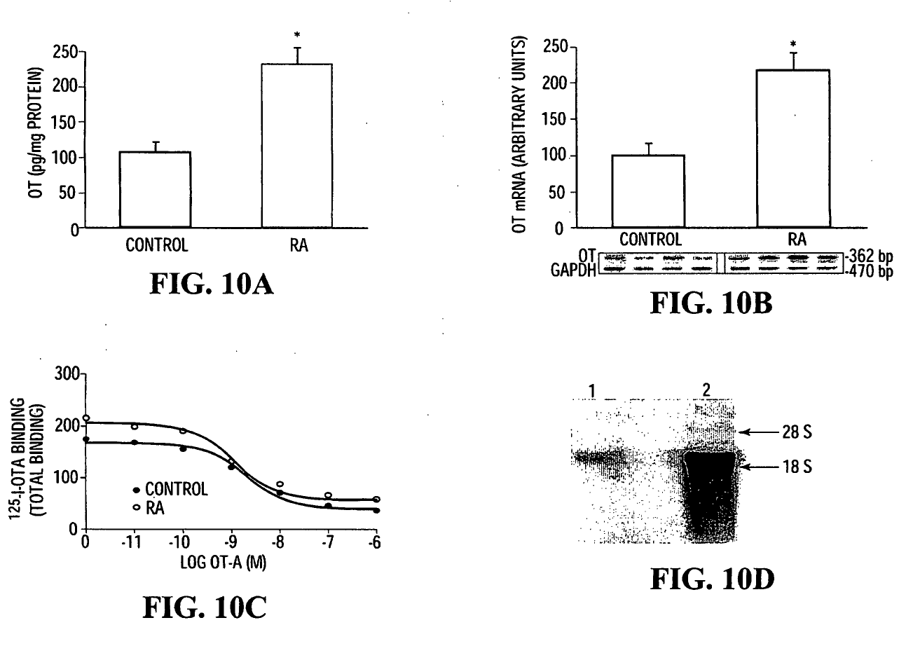

The thyroid hormone T₃ was also reported to induce cardiomyogenic differentiation of P19 cells, with an optimal effective concentration of 30 nM (Rodriguez et al., 1994). Similar to the results observed with DMSO and RA-induced differentiation, OTA abolished cardiac differentiation induced by T₃ (FIG. 9). Altogether, the results obtained with OTA suggest that OT/OTR could be universally involved in mechanisms of action of cardiomyogenic substances.

P19 stem cells express oxytocin receptor as shown by RT-PCR and immunocytochemistry (Paquin et al., PNAS 2002). Preliminary results suggest that differentiation of stem cells into cardiomyocytes depends on the early potential of inducer to increase OTR. This stimulatory effect is already observed by RT-PCR in the second day of differentiation protocol. In this respect OT is a most potent OTR inducer comparing to RA and DMSO. OT antagonist (OTA) has no effect on OTR expression at this stage of differentiation.

Throughout this application, various references are referred to describe more fully the state of the art to which this invention pertains. The disclosures of these references are hereby incorporated by reference into the present disclosure.

### REFERENCES

1. Gimpl, G. & Fahrenholz, F. (2001) Physiol Rev. 81, 629-683.
2. Ashton, N. & Balment, R. J. (1991) Acta Endocrinol. (Copenh) 124, 91-97.
3. Stoneham, M. D., Everitt, B. J., Hansen, S., Lightman, S. L. & Todd, K. (1985) J Endocrinol 107, 97-106.
4. Verbalis, J. G., Mangione, M. P. & Stricker, E. M. (1991) Endocrinology 128, 1317-1322.
5. Nishimori, K., Young, L. J., Guo, Q., Wang, Z., Insel, T. R. & Matzuk, M. M. (1996) Proc Natl Acad Sci USA 93, 11699-11704.
6. Wagner, K. U., Young, W. S., III, Liu, X., Ginns, E. I., Li, M., Furth, P. A. & Hennighausen, L. (1997) Genes Funct. 1, 233-244.
7. Gutkowska, J., Jankowski, M., Lambert, C., Mukaddam-Daher, S., Zingg, H. H. & McCann, S. M. (1997) Proc Natl Acad Sci USA 94, 11704-11709.
8. Jankowski, M., Hajjar, F., Al Kawas, S., Mukaddam-Daher, S., Hoffman, G., McCann, S. M. & Gutkowska, J. (1998) Proc Natl Acad Sci USA 95, 14558-14563.
9. Cassoni, P., Sapino, A., Fortunati, N., Munaron, L., Chini, B. & Bussolati, G. (1997) Int. J. Cancer 72, 340-344.
10. Cassoni, P., Sapino, A., Stella, A., Fortunati, N. & Bussolati, G. (1998) Int. J. Cancer 77, 695-700.
11. Cassoni, P., Fulcheri, E., Carcangiu, M. L., Stella, A., Deaglio, S. & Bussolati, G. (2000) J. Pathol. 190, 470-477.
12. Copland, J. A., Ives, K. L., Simmons, D. J. & Soloff, M. S. (1999) Endocrinology 140 , 4371-4374.
13. Martens, H., Kecha, O., Charlet-Renard, C., Defresne, M. P. & Geenen, V. (1998) Neuroendocrinology 67, 282-289.
14. Geenen, V., Kecha, O., Brilot, F., Charlet-Renard, C. & Martens, H. (1999) Neuroimmunomodulation. 6, 115-125.
15. Plecas, B., Popovic, A., Jovovic, D. & Hristic, M. (1992) J. Endocrinol. Invest 15, 249-253.
16. Thibonnier, M., Conarty, D. M., Preston, J. A., Plesnicher, C. L., Dweik, R. A. & Erzurum, S. C. (1999) Endocrinology 140, 1301-1309.
17. Cassoni, P., Sapino, A., Munaron, L., Deaglio, S., Chini, B., Graziani, A., Ahmed, A. & Bussolati, G. (2001) Endocrinology 142, 1130-1136.
18. Sapino, A., Macri, L., Tonda, L. & Bussolati, G. (1993) Endocrinology 133, 838-842.
19. Chard, T., Boyd, N. R., Forsling, M. L., McNeilly, A. S. & Landon, J. (1970) J Endocrinol 48, 223-234.
20. Schriefer, J. A., Lewis, P. R. & Miller, J. W. (1982) Biol Reprod 27, 362-368.
21. Widmer, H., Durroux, T., Kempf, H., Mouillac, B., Gasc, J. M. & Barberis, C. (1999) Abstracts of 1999 World Congress on Neurohypophysial Hormones 94.
22. Haanwinckel, M. A., Elias, L. K., Favaretto, A. L., Gutkowska, J., McCann, S. M. & Antunes-Rodrigues, J. (1995) Proc Natl Acad Sci USA 92, 7902-7906.
23. Favaretto, A. L., Ballejo, G. O., Albuquerque-Araujo, W. I., Gutkowska, J., Antunes-Rodrigues, J. & McCann, S. M. (1997) Peptides 18, 1377-1381.
24. Mukaddam-Daher, S., Lin, Y. L., Gutkowska, J. & Cardinal, R. (2001) Hypertension.
25. Cameron, V. A., Aitken, G. D., Ellmers, L. J., Kennedy, M. A. & Espiner, E. A. (1996) Endocrinology 137, 817-824.
26. Gutkowska, J., Bhat, P., Wang, D., Mukaddam-Daher, S., McCann, S. M. & Jankowski, M. (2002) 19th Scientific meeting of the international society of hypertension, Prague, June 2002 (Abstract).
27. McBurney, M. W. (1993) Int J Dev Biol 37, 135-140.
28. Rudnicki, M. A. & McBurney, M. W. (1987) in Teratocarcinomas and embryonic stem cells: a practical approach., ed. Robertson, E. J. (IRL Press, Oxford, UK), pp. 19-49.
29. Laplante, I., Paquin, J. & Beliveau, R. (2001) Brain Res. Dev. Brain Res. 129, 157-168.
30. Jeannotte, R., Paquin, J., Petit-Turcotte, C. & Day, R. (1997) DNA Cell Biol 16, 1175-1187.
31. Cadet, N. & Paquin, J. (2000) Dev Brain Res 120, 211-221.
32. Skerjanc, I. S. (1999) Trends Cardiovasc. Med. 9, 139-143.
33. Srivastava, D. & Olson, E. N. (2000) Nature 407, 221-226.
34. Seidman, C. E., Bloch, K. D., Smith, J. A. & Seidman, J. G. (1984) Science 226, 1206-1209.
35. Laemmli, U. K. (1970) Nature 227, 680-685.
36. Flucher, B. E. & Franzini-Armstrong, C. (1996) Proc. Natl. Acad. Sci. U.S.A 93, 8101-8106.
37. Summerhayes, I. C., Lampidis, T. J., Bernal, S. D., Nadakavukaren, J. J., Nadakavukaren, K. K., Shepherd, E. L. & Chen, L. B. (1982) Proc. Natl. Acad. Sci. U.S.A 79, 5292-5296.
38. Monge, J. C., Stewart, D. J. & Cernacek, P. (1995) J Biol Chem 270, 15385-15390.
39. Boer, P. H. (1994) Biochem. Biophys. Res. Commun. 199, 954-961.
40. Mohun, T. & Sparrow, D. (1997) Curr. Opin. Genet. Dev. 7, 628-633.
41. Grepin, C., Nemer, G. & Nemer, M. (1997) Development 124, 2387-2395.
42. Skerjanc, I. S., Petropoulos, H., Ridgeway, A. G. & Wilton, S. (1998) J. Biol. Chem. 273, 34904-34910.
43. Morley, P. & Whitfield, J. F. (1993) J. Cell Physiol 156, 219-225.
44. Wilton, S. & Skerjanc, I. (1999) In Vitro Cell Dev. Biol. Anim 35, 175-177.
45. Hartman, R. D., Rosella-Dampman, L. M., Emmert, S. E. & Summy-Long, J. Y. (1986) Endocrinology 119, 1-11.
46. Insel, T. R., Winslow, J. T. & Witt, D. M. (1992) Endocrinology 130, 2602-2608.
47. Di Scala-Guenot, D. & Strosser, M. T. (1995) Am. J. Physiol 268, C413-C418.
48. Furuya, K., Mizumoto, Y., Makimura, N., Mitsui, C., Murakami, M., Tokuoka, S., Ishikawa, N., Nagata, I., Kimura, T. & Ivell, R. (1995) Adv. Exp. Med. Biol. 395, 523-528.
49. Shinozuka, N., Yen, A. & Nathanielsz, P. W. (2000) Am. J. Physiol Heart Circ. Physiol 278, H41-H49.
50. Breton, C., Haenggeli, C., Barberis, C., Heitz, F., Bader, C. R., Bernheim, L. & Tribollet, E. (2002) J. Clin. Endocrinol. Metab 87, 1415-1418.
51. Beltrami, A. P., Urbanek, K., Kajstura, J., Yan, S. M., Finato, N., Bussani, R., Nadal-Ginard, B., Silvestri, F., Leri, A., Beltrami, C. A. et al. (2001) N. Engl. J. Med. 344, 1750-1757.
52. Condorelli, G., Borello, U., De Angelis, L., Latronico, M., Sirabella, D., Coletta, M., Galli, R., Balconi, G., Follenzi, A., Frati, G. et al. (2001) Proc. Natl. Acad. Sci. U.S.A 98, 10733-10738.
53. Jackson, K. A., Majka, S. M., Wang, H., Pocius, J., Hartley, C. J., Majesky, M. W., Entman, M. L., Michael, L. H., Hirschi, K. K. & Goodell, M. A. (2001) J. Clin. Invest 107, 1395-1402.
54. Rodriguez E R, Tan C D, Onwuta U S, Parrillo J E. 1994. Cardiac myocyte differentiation induced by 3,5,3′-triiodo-L-thyronine (T3) in P19 teratocarcinoma cells is accompanied by preferential binding of RGG(T/A)CA direct repeats spaced by 4 base pairs in the DNA. Biochem. Biohys. Res. Commun. 205:1899-1906).

Although preferred embodiments of the present invention have been described in detail herein and illustrated in the accompanying drawings, it is to be understood that the invention is not limited to these precise embodiments and that various changes and modifications may be effected therein without departing from the scope or spirit of the present invention.

1. A method of inducing differentiation of a non-cardiomyocyte into a cardiomyocyte, said method comprising stimulating oxytocin receptor (OTR) activity in said non-cardiomyocyte.

2. The method of claim 1, wherein said method comprises contacting said non-cardiomyocyte with an agent capable of stimulating OTR activity.

3. The method of claim 2, wherein said agent is selected from the group consisting of oxytocin or a functional derivative thereof, retinoic acid and triiodothyronine (T₃).

4. The method of claim 3, wherein said oxytocin or functional derivative thereof has the structure: 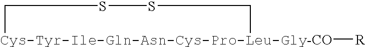
wherein R is selected from the group consisting of OH, NH₂, Gly, Gly-Lys and Gly-Lys-Arg.

5. The method of claim 1, wherein the method comprises introducing into the non-cardiomyocyte a nucleic acid capable of encoding oxytocin or an oxytocin-related compound.

6. The method of claim 5, wherein the nucleic acid is selected from the group consisting of:
(a) SEQ ID NO:5;
(b) a nucleic acid sequence capable of encoding SEQ ID NO:6; and
(c) a nucleic acid sequence substantially identical to (a) or (b).

7. The method of claim 1, wherein said non-cardiomyocyte is a stem or progenitor cell.

8. The method of claim 7, wherein said stem or progenitor cell is selected from the group consisting of embryonic and adult stem or progenitor cells.

9. The method of claim 7, wherein said stem or progenitor cell is selected from the group consisting of circulating and non-circulating stem or progenitor cells.

10. The method of claim 7, wherein said method is performed in vitro.

11. The method of claim 7, wherein said method is performed in vivo.

12. The method of claim 1, wherein said cardiomyocyte is characterized by an alteration of a phenotypic feature relative to said non-cardiomyocyte, wherein said phenotypic feature is selected from the group consisting of:
(a) level of oxytocin receptor (OTR) protein or OTR-encoding nucleic acid;
(b) level of ANP protein or ANP-encoding nucleic acid;
(c) level of muscular MHC protein or muscular MHC-encoding nucleic acid;
(d) level of DHPR-alphal protein or DHPR-alphal-encoding nucleic acid;
(e) level of sarcomeric marker proteins;
(f) level of ion channels;
(g) mitochondrial dye retention;
(h) appearance of rhythmic beats; and
(i) chronotropic responses.

13. A method of treating a disease characterized by cardiomyocyte loss or deficiency in an animal, said method comprising stimulating oxytocin receptor (OTR) activity in a non-cardiomyocyte cell of said animal.

14. The method of claim 13, wherein said method comprises administering an agent capable of stimulating OTR activity to said animal.

15. The method of claim 14, wherein said agent is selected from the group consisting of oxytocin or a functional derivative thereof, retinoic acid and triiodothyronine (T₃).

16. The method of claim 15, wherein said oxytocin or functional derivative thereof has the structure: 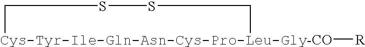
wherein R is selected from the group consisting of OH, NH₂, Gly, Gly-Lys and Gly-Lys-Arg.

17. The method of claim 15, wherein the method comprises administering a nucleic acid capable of encoding oxytocin or a functional derivative thereof to said animal.

18. The method of claim 17, wherein the nucleic acid is selected from the group consisting of:
(a) SEQ ID NO:5;
(b) a nucleic acid sequence capable of encoding SEQ ID NO:6; and
(c) a nucleic acid sequence substantially identical to (a) or (b).

19. The method of claim 13, wherein said non-cardiomyocyte is a stem or progenitor cell.

20. The method of claim 19, wherein said stem or progenitor cell is selected from the group consisting of circulating and non-circulating stem or progenitor cells.

21. The method of claim 13, wherein said animal is a mammal.

22. The method of claim 13, wherein said animal is a human.

23. The method of claim 13, wherein said disease is selected from the group consisting of cardiac congenital dysfunctions, aging-related heart pathologies, heart infarction, congestive heart failure and acute myocardial ischemia.

24. A method of treating a disease characterized by cardiomyocyte loss or deficiency in an animal, said method comprising:
(a) inducing, using the method of claim 1, differentiation of a non-cardiomyocyte cell into a cardiomyocyte; and
(b) implanting said cardiomyocyte into said animal.

25. The method of claim 24, wherein said animal is a mammal.

26. The method of claim 24, wherein said animal is a human.

27. The method of claim 24 where said disease is selected from the group consisting of cardiac congenital dysfunctions, aging-related heart pathologies, heart infarction, congestive heart failure and acute myocardial ischemia.

28. The method of claim 24, wherein said method comprises contacting said non-cardiomyocyte with an agent capable of stimulating OTR activity.

29. The method of claim 28, wherein said agent is selected from the group consisting of oxytocin or a functional derivative thereof, retinoic acid and triiodothyronine (T₃).

30. The method of claim 29, wherein said oxytocin or functional derivative thereof has the structure: 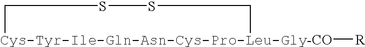
wherein R is selected from the group consisting of OH, NH₂, Gly, Gly-Lys and Gly-Lys-Arg.

31. The method of claim 24, wherein the method comprises introducing into the non-cardiomyocyte a nucleic acid capable of encoding oxytocin or a functional derivative thereof.

32. The method of claim 31, wherein the nucleic acid is selected from the group consisting of:
(a) SEQ ID NO:5;
(b) a nucleic acid sequence capable of encoding SEQ ID NO:6; and
(c) a nucleic acid sequence substantially identical to (a) or (b).

33. The method of claim 24, wherein said non-cardiomyocyte is a stem or progenitor cell.

34. The method of claim 33, wherein said stem or progenitor cell is selected from the group consisting of embryonic and adult stem or progenitor cells.

35. The method of claim 33, wherein said stem or progenitor cell is selected from the group consisting of circulating and non-circulating stem or progenitor cells.

36. The method of claim 24, wherein said non-cardiomyocyte is autologous to said animal.

37. The method of claim 36, said method further comprising obtaining said non-cardiomyocyte from said animal prior to inducing said differentiation.

38. The method of claim 24, wherein said non-cardiomyocyte is non-autologous to said animal.

39. The method of claim 38, wherein said non-cardiomyocyte is allogenic to said animal.

40. The method of claim 38, wherein said non-cardiomyocyte is xenogenic to said animal.

41. The method of claim 24, wherein said cardiomyocyte is characterized by an alteration of a phenotypic feature relative to said non-cardiomyocyte, wherein said phenotypic feature is selected from the group consisting of:
(a) level of oxytocin receptor (OTR) protein or OTR-encoding nucleic acid;
(b) level of ANP protein or ANP-encoding nucleic acid;
(c) level of muscular MHC protein or muscular MHC-encoding nucleic acid;
(d) level of DHPR-alphal protein or DHPR-alphal-encoding nucleic acid;
(e) level of sarcomeric marker proteins;
(f) level of ion channels;
(g) mitochondrial dye retention;
(h) appearance of rhythmic beats; and
(i) chronotropic responses.

42-65. (canceled)

66. A method of identifying or characterizing a compound for inducing differentiation of a non-cardiomyocyte cell into a cardiomyocyte, said method comprising:
(a) contacting a test compound with a cell comprising an oxytocin receptor (OTR) or an OTR-encoding nucleic acid; and
(b) determining whether OTR activity or expression is increased in the presence of the test compound, said increase in OTR activity or expression being an indication that the test compound may be used for inducing differentiation of a non-cardiomyocyte into a cardiomyocyte.

67. A method of identifying or characterizing a compound for treatment of a disease characterized by cardiomyocyte loss or deficiency, said method comprising:
(a) contacting a test compound with a cell comprising an oxytocin receptor (OTR) or an OTR-encoding nucleic acid; and
(b) determining whether OTR activity or expression is increased in the presence of the test compound, said increase in OTR activity or expression being an indication that the test compound may be used for for treatment of a disease characterized by cardiomyocyte loss or deficiency.

68. (canceled)

69. A method of identifying a cell capable of differentiation to a cardiomyocyte, said method comprising determining whether OTR activity or expression is present in said cell, said presence being an indication that said cell is capable of differentiation to a cardiomyocyte.
